(19) United States
(12) Patent Application Publication
(10) Pub. No.: US 2021/0388454 A1
(43) Pub. Date: Dec. 16, 2021

(54) RAPID DIAGNOSTIC SYSTEM USING TARGETED ANTISENSE OLIGONUCLEOTIDE CAPPED PLASMONIC NANOPARTICLES

(71) Applicants: University of Maryland, Baltimore, Baltimore, MD (US); University of Maryland, Baltimore County, Baltimore, MD (US)

(72) Inventors: DIPANJAN PAN, ELLICOTT CITY, MD (US); PARIKSHIT MOITRA, BALTIMORE, MD (US); MAHA MOHAMMAD SHEHADEH ALAFEEF, BALTIMORE, MD (US); KETAN DIGHE, ELLICOTT CITY, MD (US)

(21) Appl. No.: 17/325,839

(22) Filed: May 20, 2021

Related U.S. Application Data
(60) Provisional application No. 63/038,230, filed on Jun. 12, 2020.
(60) Provisional application No. 63/057,987, filed on Jul. 29, 2020.
(60) Provisional application No. 63/106,916, filed on Oct. 29, 2020.
(60) Provisional application No. 63/181,599, filed on Apr. 29, 2021.

(51) Int. Cl. C12Q 1/70

(52) U.S. Cl. CPC C12Q 1/701; C12Q 1/707

(57) ABSTRACT

The present disclosure relates to a nanotechnology-based molecular sensing system, compositions, and methods that can be adapted to accurately detect a target gene in clinical samples, using anti-sense oligonucleotide capped plasmonic nanoparticles for selective detection of biological pathogens.

### CROSS REFERENCE TO RELATED APPLICATIONS

This application claims the benefit of and priority from U.S. Provisional Application No. 63/038,230 filed on Jun. 12, 2020, U.S. Provisional Application No. 63/057,987 filed on Jul. 29, 2020, U.S. Provisional Application No. 63/106,916 filed on Oct. 29, 2020, and U.S. Provisional Application No. 63/181,599 filed on Apr. 29, 2021, the entire contents of which are fully incorporated herein by reference.

### GOVERNMENT SUPPORT

This invention was made with government support under Grant Number EB028026 awarded by the National Institutes of Health. The government has certain rights in the invention.

### FIELD OF THE INVENTION

The invention relates to systems, compositions and methods for detection of biological pathogens. In particular, the invention relates to detection of targeted nucleic acid sequences.

### BACKGROUND OF THE INVENTION

Rapid identification of specific nucleic acid sequences has enormous potential in disease diagnosis and management. One of the most common applications of nucleic acid detection is molecular-based diagnostic tests or nucleic acid testing for the diagnosis of various infectious diseases caused by different microbes and pathogens.

Nucleic acid testing (NAT) or nucleic acid amplification testing (NAAT) is a process that involves amplification and identification of nucleic acids for diagnosis and/or guidance of therapy. Commercially available nucleic acid detection methodologies typically involve amplification of nucleic acid extracted from the bio-fluids collected from the patients under observation.

Globally, infectious diseases have an enormous impact on the community from a social as well as economic standpoint. For example, according to the World Health Organization, as of May 2021 more than 150 million people worldwide have been infected with severe acute respiratory syndrome coronavirus 2 (SARS-CoV-2), the virus that is responsible for the COVID-19 pandemic, leading to more than 3 million deaths and substantial socio-economic disruption.

A crucial shortcoming of the healthcare systems across the globe has been the ability to rapidly and accurately diagnose the disease, with contributing factors that include a shortage of test kits and specimen materials, availability of personal protective equipment and reagents, and limited certified testing centers. Further, the lack of rapid diagnostic tests along with the inaccessibility of the advanced instrumental techniques to all the diagnostic centers, especially the remote ones, contributes to the confusion surrounding which individuals should be quarantined, limits epidemiological data, and hinders tracking of pathogen transmission within as well as across communities.

The ability to perform pervasive testing has already shown benefits to countries such as South Korea and Singapore, providing precise information about mandatory quarantine for a carrier of the virus and rigorous contact tracing which in turn results in greater control in slowing the spread of the disease. Downmodulating the infection rate will therefore help to minimize the risk of overwhelming health facilities.

Current standard practice for detecting an active COVID-19 infection uses chest X-ray, chest CT, or reverse transcriptase real-time polymerase chain reaction (RT-PCR) which requires labor-intensive, laboratory-based protocols for lysis, viral RNA isolation, and removal of inhibiting materials. On the basis of this technique, numerous laboratories have developed experimental protocols using quantitative RT-PCR (qRT-PCR) methods for virus identification within 4 to 6 hours, including a test developed by U.S. Centers for Disease Control and Prevention (CDC) and approved under emergency use authorization (EUA) process. However, previously described testing protocols using RT-PCR often cannot report positive COVID-19 cases at its initial presentation, due to limitations of sample collection and transportation. Furthermore, traveling to a clinical setting for testing increases the risk of spreading the SARS-CoV-2 virus which further adds strain to a resource-limited healthcare system. These restrictions become major hurdles for situations with resource scarcity.

Additionally, while serological tests are rapid, point-of-care (POC), and require minimal equipment, their efficacy may be limited in the diagnosis of acute SARS-CoV-2 infection, as it may take several days to weeks after the onset of the symptom for a patient to develop a detectable antibody response.

Further, several variants of the SARS-CoV-2 virus have proved particularly concerning, because they have been observed to be more readily transmissible, and public health officials are concerned that vaccines may not be fully effective against such variants. Variants have resulted in additional lockdowns within many countries and restrictions on international travel, in attempts to curb the spread of variants.

Therefore, is an urgent need for other approaches that are low-cost, rapid and provide diagnosis at the POC level. Therefore, new solutions and methodologies for nucleic acid detection are in high demand.

One method of detecting SARS-CoV-2 at point-of-care is loop-mediated isothermal amplification (LAMP). At present the commercially available reverse transcriptase loop mediated isothermal amplification (RT-LAMP)-based colorimetric technologies for the diagnosis of COVID-19 typically involve isolating RNA, amplifying the desired segment and detecting the isolated genetic material. In commercial colorimetric RT-LAMP COVID-19 tests, carryover contamination is a common drawback in such reactions, which usually leads to false-positive results and decreased selectivity. Moreover, if the RT-LAMP technique is not performed following good molecular biology practices, carryover contamination can be observed in subsequent reactions leading to false-positive results. Several research groups have developed different versions of LAMP-based molecular diagnostic tests for SARS-CoV-2.

These tests have inherent limitations. For example, the Penn-RAMP method combines another isothermal amplification method with the traditional LAMP. In this case, first, a recombinase polymerase amplification (RPA) process is conducted in the cap of a test tube at 38° C. for approximately 15-20 minutes. During this RPA process, an enzyme called recombinase assists the forward outer primer (F3) and backward outer primer (B3) LAMP primers in locating the targeted sequence of the sample. In the next step, the RPA mixture is mixed with conventional LAMP reagents to detect the presence of nucleic acid. However, the technique still has certain drawbacks, such as how RPA undergoes asynchronous amplification with the potential to saturate, which can prevent accurate quantitation. Moreover, relying on two isothermal amplification approaches makes the system complicated, increases the necessary reagents, and the test cost.

In another study RT-LAMP was integrated with CRISPR-Cas12 for the detection of COVID-19. This novel CRISPR-enabled diagnostic tool, called SARS-CoV-2 DNA Endonuclease-Targeted CRISPR Trans Reporter (DETECTR), uses the enzyme Cas12 after RT-LAMP to detect particular gene sequences in the amplified RNA virus and indiscriminately cleave nearby structures once complexed. However, this technique requires highly trained staff members, and can only be utilized in the lab-based hospitals where large stationary pieces of equipment, isotopes, and reagents can be accessed easily.

Though several isothermal NAA-based (especially RT-LAMP-based) colorimetric tests have been used for SARS-CoV-2 detection, these relied on the use of pH-responsive dyes to indicate the successful amplification of the target sequence. However, in such dye-based NAA detection approaches, the process also has a high occurrence of false-positive results, owing to the prevalence of spurious amplification products.

Nanotechnology-based colorimetric bioassays are convenient and attractive in biosensor design for their simplicity, visual output, and no necessity for complex instruments. In recent years, gold nanoparticle (AuNPs) have garnered incredible attention in the field of colorimetric-based biosensing applications due to their exceptional optical properties such as high extinction coefficient, localized surface plasmon resonance, and inherent photostability. They have been used in numerous colorimetric-based biosensing applications to detect a wide range of chemical and biological targets like small molecules, proteins, metal ions, and nucleic acids where the particle changes its color in response to the reactivity of the nanosized particles to the external conditions. Despite these features, however, this technique still involves the preparation of ssDNA probes and the implementation of several intermediate steps, such as time-intensive denaturation and annealing after PCR. An isothermal RT-LAMP based assay using colorimetric gold nanoparticles has also been described for visual detection of Penaeus vanmamei, an emerging viral infection in whiteleg shrimp, a commercially important species in food production, which utilized a single gold nanoparticle linked monosense ssDNA probe for detection. However, the same drawbacks present in RT-LAMP based assays for SARS-CoV-2 detection are also found in this assay.

The Covid-19 pandemic has demonstrated there is a need for tests for the identification of biological pathogens in samples, particularly for tests which (i) do not need prior extraction steps to be performed; (ii) do not demand the use of advanced equipment (e.g., centrifuge, thermocycler, etc.); (iii) do not use conventional pH sensitive dyes or fluorescence labeling for detection; (iv) use low-cost and easily accessible reagents that can be rapidly manufactured in bulk; and/or (v) have a short turnaround time.

While the need for tests to rapidly, selectively, and efficiently identify and detect biological pathogens in samples has been aptly demonstrated in the Covid-19 pandemic, the need for such tests goes beyond the detection of SARS-CoV-2. There is a need for improved tests to detect biological pathogens across many therapeutic categories, including but not limited to detection of biological pathogens which are related to pandemic disease (including for instance the presence of biological pathogens related to outbreaks of Covid-19, SARS, MERS, Ebola, or Bird Flu), biological pathogens related to respiratory disease (including for instance influenza A or B, Streptococcus, tonsillitis, pharyngitis, or adenovirus), biological pathogens related to sexually transmitted infections (including herpes, gonorrhea, syphilis, or chlamydia), and biological pathogens related to gynecological infections (including HPV, UTI, bacterial vaginosis, and trichomonas).

Therefore, as some of the existing techniques remain laborious and technically challenging, there is an urgent unmet need for a rapid, cost-effective, and selective diagnostic test for biological pathogens that can provide fast and accurate test results within a duration of less than an hour at the high end and within minutes at the low end.

### SUMMARY OF THE INVENTION

Disclosed herein are systems, compositions, and methods for a nanotechnology-based sensing system to detect biological pathogens.

Compositions for use in the detection of a biological pathogen are described herein. In one embodiment, there is provided a composition for use in the detection of a biological pathogen in a sample is provided, the composition comprising: a plurality of first anti-sense oligonucleotides, functionalized with a thiol moiety at their 5′ ends, the sequence of which is complementary to a first nucleic acid sequence in a target gene of the biological pathogen; a plurality of second anti-sense oligonucleotides, functionalized with a thiol moiety at their 3′ ends, the sequence of which is complementary to a second nucleic acid sequence in the target gene of the biological pathogen near to the first nucleic acid sequence; and a plurality of plasmonic nanoparticles capable of covalently binding to the thiol moieties, wherein upon the first and second anti-sense oligonucleotides binding to the first and second nucleic acid sequences in the target gene respectively, the plasmonic nanoparticles covalently bind to the thiol moieties on the first and second anti-sense oligonucleotides respectively and are brought within proximity of one another and agglomerate. In another embodiment, the composition further comprises a plurality of third anti-sense oligonucleotide, functionalized with a thiol moiety at its 5′ end, the sequence of which is complementary to a third nucleic acid sequence in the target gene of the biological pathogen; and a plurality fourth anti-sense oligonucleotide, functionalized with a thiol moiety at its 3′ end, the sequence of which is complementary to a fourth nucleic acid sequence in the target gene of the biological pathogen near to the third nucleic acid sequence; wherein the third and fourth nucleic acid sequences are distant from the first and second nucleic acid sequences in the target gene, and wherein upon the third and fourth anti-sense oligonucleotides binding to the third and fourth nucleic acid sequences in the target gene respectively, the plasmonic nanoparticles covalently bind to the thiol moieties on the third and fourth anti-sense oligonucleotides and are brought within proximity of one another and agglomerate. In another embodiment, the composition is for use in the detection of SARS-CoV-2, wherein the sequences of the first, second, third and fourth anti-sense oligonucleotides are SEQ ID NO 6, SEQ ID NO 7, SEQ ID NO 8, and SEQ ID NO 9, respectively. In another embodiment, the plasmonic nanoparticles are gold nanoparticles. In another embodiment, he first and second anti-sense oligonucleotides have an unpaired probability for the first and second nucleic acid sequence respectively of at least 0.5. In another embodiment, the first and second anti-sense oligonucleotides have a binding of energy of less than −8 kcal/mol. In another embodiment, the first and second anti-sense oligonucleotides are present in differing ratios relative to the plasmonic nanoparticles.

In one embodiment, there is provided a diagnostic apparatus for the detection of a biological pathogen in a clinical sample, comprising: a container for a clinical sample in solution; a plurality of first anti-sense oligonucleotides, functionalized with a thiol moiety at their 5′ ends, the sequence of which is complementary to a first nucleic acid sequence in a target gene of the biological pathogen; a plurality of second anti-sense oligonucleotides, functionalized with a thiol moiety at their 3′ ends, the sequence of which is complementary to a second nucleic acid sequence in the target gene of the biological pathogen near to the first nucleic acid sequence; and a plurality of plasmonic nanoparticles capable of covalently binding to the thiol moieties; wherein if the clinical sample contains the biological pathogen, upon mixing the first and second anti-sense oligonucleotides and plasmonic nanoparticles with the clinical sample in solution, the first and second anti-sense oligonucleotides bind to the first and second nucleic acid sequences in the target gene respectively, and the plasmonic nanoparticles covalently bind to the thiol moieties on the first and second anti-sense oligonucleotides respectively and are brought within proximity of one another and agglomerate. In another embodiment, the apparatus further comprises a plurality of third anti-sense oligonucleotide, functionalized with a thiol moiety at their 5′ ends, the sequence of which is complementary to a third nucleic acid sequence in the target gene of the biological pathogen; and a plurality fourth anti-sense oligonucleotide, functionalized with a thiol moiety at their 3′ ends, the sequence of which is complementary to a fourth nucleic acid sequence in the target gene of the biological pathogen near to the third nucleic acid sequence; wherein the third and fourth nucleic acid sequences are distant from the first and second nucleic acid sequences in the target gene, and wherein if the clinical sample contains the biological pathogen, upon mixing the third and fourth anti-sense oligonucleotides and plasmonic nanoparticles with the clinical sample in solution, the third and fourth anti-sense oligonucleotides bind to the third and fourth nucleic acid sequences in the target gene respectively, and the plasmonic nanoparticles covalently bind to the thiol moieties on the third and fourth anti-sense oligonucleotides and are brought within proximity of one another and agglomerate. In another embodiment, the apparatus is for use in the detection of SARS-CoV-2, wherein the sequences of the first, second, third and fourth anti-sense oligonucleotides are SEQ ID NO 6, SEQ ID NO 7, SEQ ID NO 8, and SEQ ID NO 9, respectively. In another embodiment, the plasmonic nanoparticles are gold nanoparticles. In another embodiment, the agglomeration is detectable through a color change in the solution, further comprising a colorimeter to detect the color change. In another embodiment, the apparatus further comprises a nuclease enzyme, wherein when the nuclease enzyme is mixed with the anti-sense oligonucleotides and plasmonic nanoparticles with a clinical sample containing the biological pathogen in solution, the nuclease enzyme cleaves the hybrid anti-sense oligonucleotide and nucleic acid sequence from the remainder of the target gene, resulting in the agglomeration and precipitation of covalently bound plasmonic nanoparticles from solution. In another embodiment, the first and second anti-sense oligonucleotides are present in differing ratios relative to the plasmonic nanoparticles.

In one embodiment, there is provided a method for detecting a biological pathogen in a clinical sample, the method comprising: collecting a clinical sample in solution; mixing a plurality of first anti-sense oligonucleotides, functionalized with a thiol moiety at their 5′ ends, the sequence of which is complementary to a first nucleic acid sequence in a target gene of the biological pathogen, with the clinical sample in solution; mixing a plurality of second anti-sense oligonucleotides, functionalized with a thiol moiety at their 3′ ends, the sequence of which is complementary to a second nucleic acid sequence in the target gene of the biological pathogen near to the first nucleic acid sequence, with the clinical sample in solution; and mixing a plurality of plasmonic nanoparticles capable of covalently binding to the thiol moieties with the clinical sample in solution; wherein if the clinical sample contains the biological pathogen, upon mixing the first and second anti-sense oligonucleotides and plasmonic nanoparticles with the clinical sample in solution, the first and second anti-sense oligonucleotides bind to the first and second nucleic acid sequences in the target gene respectively, and the plasmonic nanoparticles covalently bind to the thiol moieties on the first and second anti-sense oligonucleotides respectively and are brought within proximity of one another and agglomerate. In another embodiment, the method further comprises: mixing a plurality of third anti-sense oligonucleotide, functionalized with a thiol moiety at their 5′ ends, the sequence of which is complementary to a third nucleic acid sequence in the target gene of the biological pathogen, with the clinical sample in solution; and mixing a plurality fourth anti-sense oligonucleotide, functionalized with a thiol moiety at their 3′ ends, the sequence of which is complementary to a fourth nucleic acid sequence in the target gene of the biological pathogen near to the third nucleic acid sequence, with the clinical sample in solution; wherein the third and fourth nucleic acid sequences are distant from the first and second nucleic acid sequences in the target gene, and wherein if the clinical sample contains the biological pathogen, upon mixing the third and fourth anti-sense oligonucleotides and plasmonic nanoparticles with the clinical sample in solution, the third and fourth anti-sense oligonucleotides bind to the third and fourth nucleic acid sequences in the target gene respectively, and the plasmonic nanoparticles covalently bind to the thiol moieties on the third and fourth anti-sense oligonucleotides and are brought within proximity of one another and agglomerate. In another embodiment, the method is for detecting SARS-CoV-2, wherein the sequences of the first, second, third and fourth anti-sense oligonucleotides are SEQ ID NO 6, SEQ ID NO 7, SEQ ID NO 8, and SEQ ID NO 9, respectively. In another embodiment, the plasmonic nanoparticles are gold nanoparticles. In another embodiment, the method further comprises performing nucleic acid amplification of the clinical sample in solution prior to the mixing of the plurality of first anti-sense oligonucleotides therewith. In another embodiment, the nucleic acid amplification is performed by loop-mediated isothermal amplification.

### BRIEF DESCRIPTION OF THE DRAWINGS

FIG. 1 shows a schematic representation of colorimetric methods of detection of the agglomeration of ASO-capped AuNPs.

FIG. 2A shows differentially functionalized ASOs with their sequences. FIG. 2B shows a schematic representation of the proposed concept behind the agglomeration of gold nanoparticles, when capped with the ASOs.

FIG. 3A shows the Transmission Electron Microscopy (TEM) image of ASO-capped AuNPs. FIG. 3B shows zoom-in TEM images of Au-ASOₘᵢₓ nanoparticles with the individual ASO-capped AuNPs shown as insets. FIG. 3C shows the comparative change in hydrodynamic diameter of individual ASO-capped AuNPs and the mixture of four ASO-capped AuNPs. FIG. 3D shows the normalized change in absorbance of AuNPs before and after addition of thiol modified ASOs.

FIG. 4A shows the increase in absorbance at 660 nm for AuNPs capped with different concentrations of ASO1. FIG. 4B shows the increase in absorbance at 660 nm for AuNPs capped with different concentrations of ASO2. FIG. 4C shows the increase in absorbance at 660 nm for AuNPs capped with different concentrations of ASO3. FIG. 4D shows the increase in absorbance at 660 nm for AuNPs capped with different concentrations of ASO4.

FIG. 5A shows the normalized change in absorbance of AuNPs before and after addition of RNA with SARS-CoV-2 viral load. FIG. 5B shows the comparative change in average hydrodynamic diameter of the Au-ASOmix before and after addition of 0.1, 0.5, and 1 ng/μL RNA with SARS-CoV-2 viral load. FIGS. 5C, 5D, 5E, and 5F show TEM images of the Au-ASOmix after addition of RNA with SARS-CoV-2 viral load. FIG. 5G shows the percent change in absorbance at 660 nm of the ASO-capped AuNPs over incubation time with RNA containing SARS-CoV-2, with error bars indicating average results from four independent experiments performed in triplicate.

FIG. 6A shows the response of the Au-ASOmix, through absorbance at 660 nm, to RNA isolated from non-infected Vero cells, Vero cells infected with MERS-CoV, and Vero cells infected with SARS-CoV-2. FIG. 6B shows the relative change in absorbance at 660 nm for the Au-ASOmix treated with SARS-CoV-2 RNA, followed by addition of RNase H at different incubation temperatures. FIG. 6C shows a schematic representation for the visual “naked-eye” detection of SARS-CoV-2 with treatment of RNase H.

FIG. 7 shows the absorption spectrum of AuNPs capped with ASO1 and ASO2 responding to confirmed positive and negative COVID-19 samples, demonstrating a significant shift of the plasmonic peak at about 520 nm in the presence of SARS-CoV-2.

FIG. 8 shows a schematic representation of the selective visual detection of SARS-CoV-2 RNA mediated by the suitably designed ASO-capped AuNPs.

FIG. 9 shows the agarose gel electrophoresis of reaction mixtures after nucleic acid amplification.

FIG. 10A shows the color change in response to RNA extracted from clinical nasopharyngeal and oropharyngeal swab samples of subjects who are positive or negative for COVID-19. FIG. 10B shows the absorbance of the reaction mixture with 29 COVID-19 positive clinical samples and 32 COVID-19 negative clinical samples. Clinical samples are identified as positive or negative for COVID-19 by RT-PCR.

FIG. 11A shows the color change in response to artificial saliva samples spiked with COVID-19 positive RNA, COVID-19 negative RNA, and MERS-CoV RNA. FIG. 11B shows the absorbance of the reaction mixture for 60 samples spiked with COVID-19 positive RNA and 30 samples spiked with COVID-19 negative RNA.

FIG. 12A shows the color change in response to clinical samples either positive or negative for COVID-19, tested without prior RNA extraction or purification. FIG. 12B shows the absorbance of the reaction mixture with 11 COVID-19 positive clinical samples and 11 COVID-19 negative clinical samples tested without prior RNA extraction or purification. Clinical samples are identified as positive or negative for COVID-19 by RT-PCR.

FIG. 13A shows the response of Au-ASOmix comprising ASO5 and ASO6, through absorbance at 520 nm and 650 nm, in three representative clinical samples positive for hepatitis C virus (HCV). FIG. 13B shows the color change in response to exposure to clinical samples which are either negative or positive for HCV.

FIG. 14 shows the response of Au-ASOmix comprising ASO7 and ASO8, as relative change in sensor output in response to lysates from cells infected with MERS-CoV and Influenza B H1N1, Influenza A H1N1 Maryland strain, SARS-CoV, and SARS-CoV-2.

### DETAILED DESCRIPTION OF THE INVENTION

This disclosure, and the embodiments described herein, are provided by way of illustration of an example, or examples, of particular embodiments of the principles of the present invention. These examples are provided for the purposes of explanation, and not limitation, of those principles and of the invention. It will be appreciated that numerous specific details have been provided for a thorough understanding of the exemplary embodiments described herein. However, it will be understood by those of ordinary skill in the art that the embodiments described herein may be practiced without these specific details. In other instances, well-known methods, procedures, apparatus, equipment and components have not been described in detail so as not to obscure the embodiments described herein. Furthermore, this description is not to be considered so that it may limit the scope of the embodiments described herein in any way, but rather as merely describing the implementation of the various embodiments described herein.

### Definitions

The terminology used herein is for the purpose of describing particular embodiments only and is not intended to be limiting of example embodiments of the invention. Terms and phrases used in this specification have their ordinary meanings as one of skill in the art would understand.

As used herein, “ASO” means anti-sense oligonucleotide. “Thiol-modified” means an oligonucleotide which has been functionalized to have a thiol moiety at either its 5′ or 3′ end. “Nanoparticles” means a particle of matter less than 500 nanometers in diameter. “Plasmonic” nanoparticle means particles, including noble metal particles, whose electron density can couple with electromagnetic radiation of wavelengths that are far larger than the particle due to the nature of the dielectric-metal interface between the medium and the particles, which exhibit scattering, absorbance, and coupling properties based on the particle geometries and relative positions. “AuNP” means one of the class of plasmonic gold nanoparticles. “Capped” nanoparticle means a nanoparticle which is covalently bound to another molecule, such as a thiol-modified ASO.

As used herein, a “biological pathogen” may include any bacteria, virus, fungus, or other organism which is capable of causing disease or deleterious health effects in a human or animal subject. As used herein, a “clinical sample” may include any sample, including a fluid or tissue sample, taken from a subject. The terms “aggregation” and “agglomeration” are generally used interchangeably herein.

### Nanotechnology-Based Molecular Sensing System

The present disclosure relates to a nanotechnology-based molecular sensing system, compositions, and methods that can be adapted to accurately detect a target gene in clinical samples, using anti-sense oligonucleotide (ASO) capped plasmonic nanoparticles for selective detection of biological pathogens. ASOs are designed to target specific gene segments of biological pathogens of interest and functionalized to conjugate with plasmonic nanoparticles. Extraction of nucleic acid is not a requirement, allowing the sensing of biological pathogens directly from clinical samples.

There is an immediate need to develop approaches that are low-cost, rapid, do not require the use of advanced equipment, and can be used as a screening tool for the diagnosis of COVID-19 infection at point-of-care (POC). Rapid, low-cost and user-friendly molecular diagnostic methods are important for combating outbreaks of infectious diseases. Especially during the current pandemic of COVID-19, it is critical to expanding the testing capacity beyond laboratory settings. There is an immediate need to develop global testing capability over existing conventional approaches. In recognition of this unmet need, the World Health Organization (WHO) has established the ASSURED criteria (Affordable, Sensitive, Specific, User-friendly, Rapid and Robust, Equipment-free, and Deliverable)—as guidelines for tests to be effective in resource-limited environments.

In certain embodiments, the nanotechnology-based molecular sensing system described herein provides one or more advantages over the alternative currently available molecular techniques. In certain embodiments, the nanotechnology-based sensing system described herein: (i) does not need prior RNA extraction; (ii) does not demand the use of advanced equipment (e.g., centrifuge, thermocycler, etc.); (iii) does not use conventional pH sensitive dyes; (iv) may be used with low-cost and easily accessible reagents that can be rapidly manufactured in bulk for commercialization and/or (v) has a short turnaround time.

One advantage of certain embodiments of the presently described system is that the necessary materials are readily accessible (including in bulk) and relatively inexpensive, allowing for a low cost per test. For instance, certain embodiments of the system may be used to test samples without extraction of nucleic acids, thus avoiding the use of expensive commercial kits containing several steps and specialized laboratory equipment such as centrifuges. In certain embodiments, the plasmonic nanoparticles can be produced at a large scale, and in certain embodiments the antisense oligonucleotides are short ssDNA sequences that are relatively economical to synthesize. In certain embodiments, the presently described system provides for rapid turnaround time to detection of a biological pathogen. With respect to the turnaround time for detection using the system described herein, in certain embodiments once suitable ASOs have been designed and chosen as described herein, detection of a biological pathogen of interest may be performed in between about 5 minutes to about 45 minutes. In certain embodiments the detection of a biological pathogen of interest may be performed in about 5, 10, 15, 20, 25, 30, 35, 40 or 45 minutes. In one embodiment, isothermal amplification of the target genetic material may be completed within about 35 minutes, and colorimetric detection of the target genetic material from a biological pathogen of interest may be completed within about 5 minutes, providing for a total time of detection of less than about 40 minutes from isolation of sample material. In another embodiment, upon extraction of the target genetic material within a sample, “naked-eye” detection of the target genetic material from a biological pathogen of interest may be performed following an incubation of about 5 minutes.

The system disclosed herein may be adapted to detect target genes of various biological pathogens of interest. This detection may be performed directly in clinical samples. For example, the system may be adapted to detect biological pathogens which are related to pandemic disease, including for instance biological pathogens related to outbreaks of Covid-19, SARS, MERS, Ebola, or Bird Flu. The system may be adapted to detect biological pathogens related to respiratory disease, including for instance influenza A or B, Streptococcus, or adenovirus, or pathogens related to tonsillitis or pharyngitis. The system may be adapted to detect biological pathogens related to sexually transmitted infections, including herpes, gonorrhea, syphilis, or chlamydia, or biological pathogens related to gynecological infections including HPV, UTI, bacterial vaginosis, and trichomonas.

### Anti-Sense Oligonucleotides

ASOs may be designed to target at least one gene of interest. Suitable ASOs are selected based on their target binding energy and disruption energy. Pairs of ASOs may be chosen that bind to closely spaced regions in the target sequences. ASOs are functionalized at either the 5′ or 3′ end, such that they are capable of binding plasmonic nanoparticles. In certain embodiments, ASOs are functionalized with a thiol moiety either at the 5′ or 3′ end and then used to cap plasmonic nanoparticles. In certain embodiments, the target gene may be present as ssDNA, dsDNA, or RNA.

In certain embodiments, ASOs of a preferred length of 20 nucleotide bases are chosen. In one embodiment, ASOs are chosen using software for statistical folding of nucleic acids. In one such embodiment, the following criteria are used: (i) guanine and cysteine percent content (GC %) within 40-60%; (ii) target sequences with GGGG are excluded; (iii) average unpaired probability of the ASOs for target site nucleotides is set to ≥0.5; (iv) all sites targeted to the peak in the accessibility profile are ranked by their average unpaired probability (the higher the better), with the threshold probability above 0.5; (v) the top 20 candidate ASOs with the highest average unpaired probability are selected for further consideration; and (vi) the binding energy of the ASOs is compared with the target sequence and the binding energy cutoff for the selection of ASOs is set at ≤−8 kcal/mol. From candidate ASOs, ASOs are then chosen based on their comparative binding disruption energies and binding energies with the target sequence.

In one embodiment, ASOs are chosen in pairs binding to closely spaced regions in the target sequence, and each ASO in such a pair is differently functionalized such that the functionalized ends come close to each other when the two ASOs are bound to the target sequence. As the functionalized ends are bound to plasmonic nanoparticles, they cause the plasmonic nanoparticles to come close to each other only in the presence of the target sequence leading to agglomeration among nanoparticles which can be observed by the change in the surface plasmon band. In one embodiment, the plasmonic nanoparticles are gold nanoparticles. In one embodiment, multiple such pairs of ASOs are chosen, covering multiple regions of a target gene at the same time. One advantage of using multiple pairs of ASOs, covering multiple regions of a target gene is accurate recognition of a target sequence even if other genomic segments of the biological pathogen are subject to mutation. In one embodiment, two such pairs of ASOs are chosen, covering two regions of a target gene at the same time.

In certain embodiments, the differentially functionalized ends of the ASOs are utilized to exchange the surface capping agent of the plasmonic nanoparticles. In one embodiment, differentially functionalized thiol modified ASOs are utilized to exchange the surface capping agent of citrate stabilized gold nanoparticles. In certain embodiments, ASO capped gold nanoparticles exhibit a small anhydrous size of <30 nm, which are well dispersed without the formation of a large entity. In certain embodiments, the average hydrodynamic sizes of the individual ASO capped gold nanoparticles is less than 60 nm. In certain embodiments, the formation of ASO conjugated thiol stabilized gold nanoparticles may be confirmed from their surface plasmon bands. In certain embodiments, two absorption peaks, one at ˜530 nm and the other at ˜620 nm, may be observed after the thiol modified ASO capping on the surface of the gold nanoparticles.

In certain embodiments, the relative sensitivity of the various ASO capped gold nanoparticles towards the target gene may be monitored with the comparative increase in absorbance at 660 nm. The surface capping of thiol-modified ASOs together with the comparative ratio of the ASOs to the plasmonic nanoparticles play a major role in determining the sensitivity of the plasmonic nanoparticles toward the target gene. The optimal ratio of ASOs to plasmonic nanoparticles during functionalization of the ASOs varies according to the selected ASO. In certain embodiments, during functionalization of the ASOs, the plasmonic nanoparticles are citrate stabilized gold nanoparticles present at a concentration of about 3×10¹⁰ particles/mL, and each ASO is independently present at concentrations of about 0.5 μM, about 1 μM, or about 2 μM.

In one embodiment, multiple ASO capped gold nanoparticles are mixed in an equivalent amount with each other to further improve the analytical sensitivity of the gold nanoparticles towards the target gene. In one embodiment, two pairs of ASO capped gold nanoparticles, selected and modified such that the gold nanoparticles come close to each other only in the presence of the target sequence, are mixed in an equivalent amount with each other to further improve the analytical sensitivity of the gold nanoparticles towards the target gene.

### Detection of Target Genes Using ASO Capped Plasmonic Nanoparticles

In certain embodiments, the ASO capped plasmonic nanoparticles are individually dispersed in solution in absence of the target gene, but when in the presence of the target gene tend to agglomerate forming large clusters. In certain embodiments, the ASO capped plasmonic nanoparticles are ASO capped gold nanoparticles.

In certain embodiments, sensitivity of detection is improved when ASOs are chosen in pairs binding to closely spaced regions in the target sequence, allowing the functionalized ends to come close to each other when the two ASOs are bound to the target sequence. As the thiol groups are conjugated to plasmonic nanoparticles, they cause the plasmonic nanoparticles to come close to each other in the presence of the target sequence leading to agglomeration among nanoparticles at an increased rate relative to plasmonic nanoparticles bound to thiol groups on ASOs which are not chosen in pairs.

The aggregation of plasmonic nanoparticles may be confirmed using techniques known in the art. For example, in one embodiment, aggregation of plasmonic nanoparticles may be confirmed by a change in optical properties, such as surface plasmon resonance. In one embodiment, the plasmonic nanoparticles are gold nanoparticles, and aggregation of gold nanoparticles may be confirmed from the increase in absorbance of the aggregation band at 660 nm, measured with a colorimeter. In one embodiment, aggregation of plasmonic nanoparticles may be confirmed from increase in hydrodynamic diameter of plasmonic nanoparticles. In one embodiment, aggregation of plasmonic nanoparticles may be confirmed from increase in average size of aggregated nanoparticles, as measured by transmission electron microscopy (TEM). In one embodiment, the aggregation of plasmonic nanoparticles may be observed by a color change in solution.

In certain embodiments, agglomeration of plasmonic nanoparticles may be confirmed by visualizing under enhanced dark-field hyperspectral imaging microscope. The hyperspectral imaging (HSI) provides a label-free detection approach, combining both imaging and spectrophotometry, that can be used to localize nanomaterial based on their hyperspectral signature. The HSI system utilizes advanced optics and computational algorithms to capture a spectrum from 400 to 1000 nm at each pixel of the image with an enhanced dark-field microscopic (EDFM). The obtained spectrum represents a signature of each individual material that can be used to confirm the identity of the materials of interest in a mixture of sample. This information can further be utilized to create an image “map” to reveal the presence and location of material of interest in the targeted sample.

In certain embodiments, the distribution and location of each type of ASO capped gold nanoparticles, in a mix of multiple ASO capped gold nanoparticles, when bound with its target gene, may be identified. In one embodiment, the hyperspectral data of each individual ASO capped gold nanoparticle is recorded and stored in the spectral library for further analysis. Using these data, an image ‘map’ may be generated to identify the location of each individual ASO capped gold nanoparticle in the hybrid cluster.

In certain embodiments, the limit of detection is about 0.18 ng/μL with a dynamic range of about 0.2-3 ng/μL. In certain embodiments, nucleic acid amplification of the sample is performed prior to detection of the target gene using ASO capped plasmonic nanoparticles, which may allow target gene detection of about 10 copies/μL.

The nucleic acid molecule containing the target gene may be cleaved from the composite hybrid of the target gene sequence and ASO capped plasmonic nanoparticle, leading to a visually detectable precipitate from solution mediated by the additional agglomeration among the plasmonic nanoparticles. In one embodiment, the target gene sequence is an RNA sequence located on an RNA strand, and RNase H is used to cleave the composite hybrid of RNA and ASO capped gold nanoparticle from the RNA strand, leading to additional agglomeration among the ASO capped gold nanoparticles.

The system disclosed herein allows for multiple methods of detecting the target gene, as described above. In certain embodiments the system disclosed herein provides for both qualitative or quantitative detection of the target gene and biological pathogen. “Naked eye” detection through color change or precipitation of agglomerates of ASO capped plasmonic nanoparticles may be used to provide a qualitative determination of whether a given sample contains the target gene, and therefore contains the biological pathogen of interest. In one embodiment, the detection of the target gene and biological pathogen is made quantitative by establishing a standard curve that correlates the target gene quantity with an optical readout. In one embodiment, the sample is heated to observe a color change.

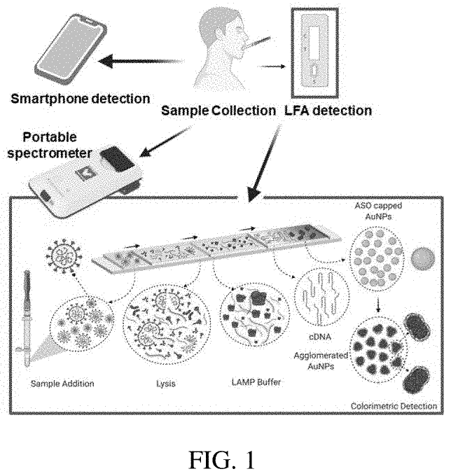

Certain embodiments of methods of detecting the target gene are shown in FIG. 1. In one embodiment, a measurement device may be configured to measure the optical indication of the sensor as a measurement of presence of the biological pathogen in a sample mixture. In one embodiment, the measurement device may be a colorimeter, a processor linked to a camera (for instance in a smartphone device), or a portable spectrometer that measures changes of the wavelength of light or intensity provided by the plasmonic nanoparticles. The measurement device may further be coupled to a processor (for instance, a computer or smartphone device) for further processing, output (for instance display or communication to another device), or local or remote storage. In one embodiment, the detection of a pathogen is performed in a laminar flow assay which allows for the performance of consecutive steps in a single device or chip. In one embodiment, such steps to be performed in a laminar flow assay device may include sample addition, cell lysis, nucleic acid amplification (for example by LAMP), and colorimetric detection of biological pathogen through agglomeration of ASO capped gold nanoparticles designed and chosen as described herein.

In certain embodiments, the ASOs may have additional components, configured to agglomerate only in the presence of a specific target sequence, such as RNA or DNA of a biological pathogen. In certain embodiments, the ASOs may be specifically chosen so as to bind to a desired component of a biological pathogen for detection.

### Sample Preparation

The ASO capped plasmonic nanoparticles may be used to detect a target gene in a clinical sample. In certain embodiments, a lysis step may be performed on the sample. Examples of lysis methods know in the art include thermal lysis, alkaline lysis, detergent lysis, and enzymatic cell lysis. In an embodiment, detergent lysis is employed, and a detergent is added to the clinical sample. Suitable detergents are known in the art, including: sodium dodecyl sulphate (SDS), Triton™ X 100, Triton™ X 114, NP-40, Tween™ 20, Tween™ 80, cetyltrimethylammonium bromide (CTAB), CHAPS and CHAPSO. In one embodiment, nucleic acids are extracted from a clinical sample prior to detection of the target gene. Methods for extraction of nucleic acids are known in the art. For example, if the nucleic acid of interest is RNA, suitable methods of RNA extraction include hot acid phenol, Qiamp DSP Virus Spin kit, Total RNA Purification Kit, RNEasy™ Mini Kit (Qiagen™), Illustra™ RNAspin Mini RNA Isolation Kit (GET™), Viral Nucleic Acid (DNA/RNA) Extraction Kit I, and EXTRAzol/TRlzol™. Alternatively, no extraction step may be performed prior to detection of the target gene.

In certain embodiments, nucleic acid amplification is performed prior to detection of the target gene. Nucleic acid amplification may be performed by methods known in the art. In one embodiment, nucleic acid amplification is performed using loop-mediated isothermal amplification (LAMP) methods. In one embodiment, the target gene is present as RNA in the biological pathogen of interest and in the clinical sample, and nucleic acid amplification is performed using reverse transcriptase loop-mediated isothermal amplification (RT-LAMP). Advantages of using LAMP methods for nucleic acid amplification over other amplification methods, such as PCR, are known in the art, and may include isothermal amplification without the use of a thermocycler, and the accommodation of a wide pH and temperature range, ability to work with non-processed samples, and flexibility of readout methods.

### Test Kit or Apparatus

The nanotechnology-based sensing system described herein may also be provided as part of a kit for the detection of one or more biological pathogens of interest. Such kits may include plasmonic nanoparticles capped with suitable ASOs designed and chosen specifically to bind a nucleic acid sequence in a target gene. In certain embodiments, the ASOs are designed and chosen in pairs to bind closely spaced nucleic acid sequences in a target gene.

Such kits may optionally include additional components, including a sample collection apparatus such as a swab or collection vial, a sample collection and/or storage and/or preservation buffer or solution, lysis buffer or detergent, components and reagents for nucleic acid extraction, components and reagents for nucleic acid amplification, and/or a detection apparatus. Examples of such optional additional components are known in the art. Examples of suitable sample collection and/or storage and/or preservation buffers or solutions include viral transport medium and PrimeStore™ MTM. Examples of suitable lysis detergents include sodium dodecyl sulphate (SDS), Triton X 100, Triton X 114, NP-40, Tween 20, Tween 80, cetyltrimethylammonium bromide (CTAB), CHAPS and CHAPSO. If the nucleic acid of interest is RNA, suitable methods of RNA extraction include hot acid phenol, Qiamp DSP Virus Spin kit, Total RNA Purification Kit, RNEasy Mini Kit (Qiagen), Illustra RNAspin Mini RNA Isolation Kit (GE), Viral Nucleic Acid (DNA/RNA) Extraction Kit I, and EXTRAzol/TRIzol. An example of suitable nucleic acid amplification reagents is WarmStart™ LAMP 2× Master Mix.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques, physics, and pharmacology, within the skill of the art. Such techniques are explained fully in the literature.

### Example 1. Design and Selection of Antisense Oligonucleotides (ASOs)

During the current spread of COVID-19 causative virus, SARS-CoV-2, scientists have discovered three regions among the SARS related viral genomes which had conserved sequences. These sequences are: (a) RdRP gene (RNA-dependent RNA polymerase gene) responsible for the open reading frame ORF1ab region, (b) E gene (envelope protein gene), and (c) N gene (nucleocapsid phosphoprotein gene).26 The analytical sensitivity of both the RdRP and E genes was demonstrated to be quite high (technical limit of detection of 3.6 and 3.9 copies per reaction), while the sensitivity for N gene observed to be weaker (8.3 copies per reaction). This leaves an enormous area for the improvement of biosensors targeted for N gene sequence of SARS-CoV-2. Statistically, a sensitive biosensor selectively targeting the N gene sequence of SARS-CoV-2 with a visual ‘naked-eye’ response without the need for access to any sophisticated instrumental techniques would greatly benefit to the current sensor development research for COVID-19. In addition to this, the analytical sensitivity of the biosensor can be improved by simultaneous targeting of multiple genetic regions within the same gene sequence, which will add to features of the biosensor. This will also increase the feasibility of the assay even if one region of the viral gene undergoes mutation during its current spread. Therefore, the N gene sequence of SARS-CoV-2 was targeted.

The N gene (nucleocapsid phosphoprotein gene) was undertaken herein as the target gene sequence for the selective detection of SARS-CoV-2 isolate 2019-nCoV/USA-WA1-A12/2020 and a set of ASOs were predicted using the following methodology.

The target N-gene sequence of SARS-CoV-2 SEQ ID NO 1 was supplied to a software for statistical folding of nucleic acids and studies of regulatory RNAs, Soligo, 57 and the ASOs were predicted maintaining the folding temperature as 37° C. and ionic conditions of 1 M sodium chloride for a preferred length of ASO as 20 nucleotide bases. The filter criteria were set as follows: [1] 40%≤GC %≤60%; [2] Elimination of target sequences with GGGG; [3] Average unpaired probability of the ASOs for target site nucleotides to be ≥0.5; [4] Considering the threshold probability of above 0.5, all sites targeted to the peak in the accessibility profile are ranked by their average unpaired probability (the higher the better); [5] Among sites satisfying criteria 1-4, the top 20 ones with the highest average unpaired probability will be considered. The average unpaired probability was also used in filter criteria 3, 4 and 5 to reduce the number of reported sites in order to optimize the disruption energy calculation in the web servers. [6] Further, the binding energy of the ASOs were also compared with the target sequence where the binding energy cutoff for the selection of ASOs was kept at ≤−8 kcal/mol.

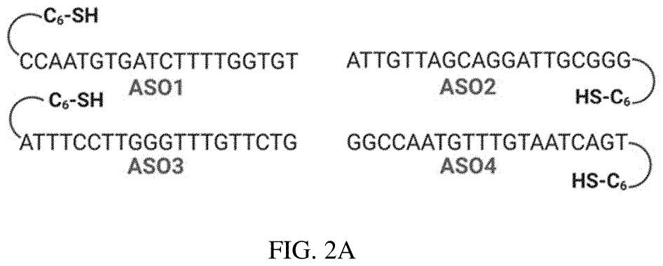

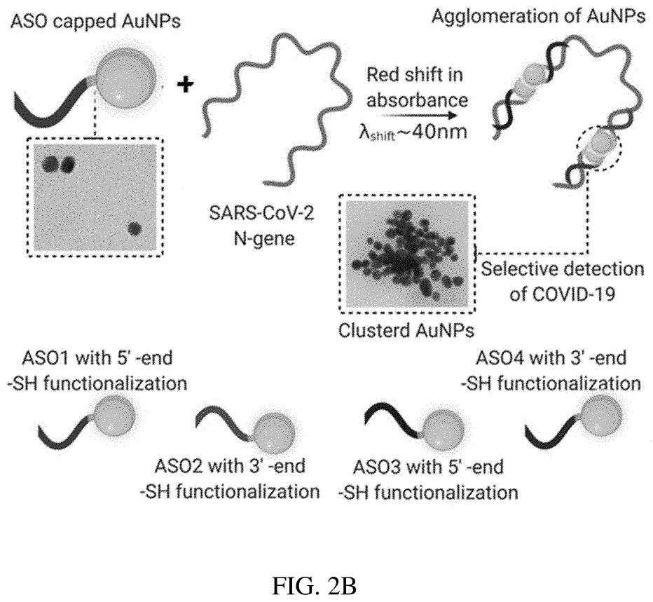

Among the predicted ASO sequences, four of the ASOs were selected based on their comparative binding disruption energies and binding energies with the target sequence (as shown in Table 1). One of the other parameters behind the selection of these four ASO sequences was their closely following target position. The ASOs were then differentially functionalized as shown in FIG. 2A: ASO1 and ASO3 were functionalized with thiol moiety at the 5′ end, whereas ASO2 and ASO4 were functionalized with thiol moiety at the 3′ end. These ASOs when used to cap gold nanoparticles are expected to get agglomerated selectively in presence of the N gene sequence of SARS-CoV-2 which can be corroborated with their complementary binding followed by aggregation propensity among the nanoparticles, as shown in FIG. 2B.

TABLE 1
Selected ASO sequences targeted for the N-gene of SARS-CoV-2.

| | | | | | Binding | |
|---|---|---|---|---|---|---|
| | | | | | site | |
| Starting | Ending | Target | | | disruption | Binding |
| target | target | sequence | Antisense oligo | GC | energy | energy |
| position | position | (5p → 3p) | (5p → 3p) | content | (kcal/mol) | (kcal/mol) |
| 421 | 440 | ACACCAAAAG | CCAATGTGATC | 40.0% | 7.6 | −15.8 |
| | | AUCACAUUGG | TTTTGGTGT | | | |
| | | (SEQ ID NO 2) | (AS01) | | | |
| | | | (SEQ ID NO 6) | | | |
| 443 | 462 | CCCGCAAUCC | ATTGTTAGCAG | 50.0% | 7.6 | −10.4 |
| | | UGCUAACAAU | GATTGCGGG | | | |
| | | (SEQ ID NO 3) | (A502) | | | |
| | | | (SEQ ID NO 7) | | | |
| 836 | 855 | CAGAACAAAC | ATTTCCTTGGG | 40.0% | 6.0 | −14.3 |
| | | CCAAGGAAAU | TTTGTTCTG | | | |
| | | (SEQ ID NO 4) | (A503) | | | |
| | | | (SEQ ID NO 8) | | | |
| 886 | 905 | ACUGAUUACA | GGCCAATGTTT | 40.0% | 8.7 | −10.0 |
| | | AACAUUGGCC | GTAATCAGT | | | |
| | | (SEQ ID NO 5) | (AS04) | | | |
| | | | (SEQ ID NO 9) | | | |

### Example 2. Synthesis of Citrate-Stabilized Gold Nanoparticles

A solution of 2.2 mM of sodium citrate was taken in milli-Q water (150 mL) and refluxed for 15 minutes under vigorous stirring. A solution of 1 mL of HAuCl4 (25 mM) was injected to the boiling solution of sodium citrate. The color of the solution changed over a time period of 20 minutes. The resulting citrate capped gold nanoparticles (AuNPs) were well suspended in H2O.

### Example 3. Functionalization of AuNPs with ASOs

Citrate stabilized AuNPs were taken at ˜3×1010 particles/mL concentration as observed through zetaview software and treated with ASOs at three different concentrations, i.e. 0.5, 1 and 2 μM from a stock of 200 μM for each of the four ASOs. The mixture was stirred at room temperature for 30 minutes, centrifuged to remove any excess of uncapped ASO from the supernatant and the pellet was then resuspended in similar volume of milli-Q water. Accordingly twelve different samples for four of the ASOs at three different concentrations were prepared and nomenclatured as Au-ASOxL, Au-ASOxM and Au-ASOxH where x defines the number of ASO as 1, 2, 3 or 4 and L, M and H are representative of low, medium and high concentrations of ASOs respectively. The nanoparticles were kept at 4° C. for future use.

### Example 4. Standardization of the ASO-Capped Gold Nanoparticle (AuNPs) for the Sensitive Detection of SARS-CoV-2

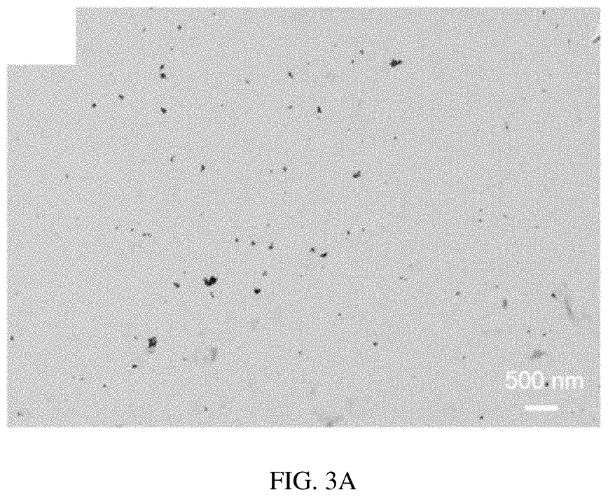

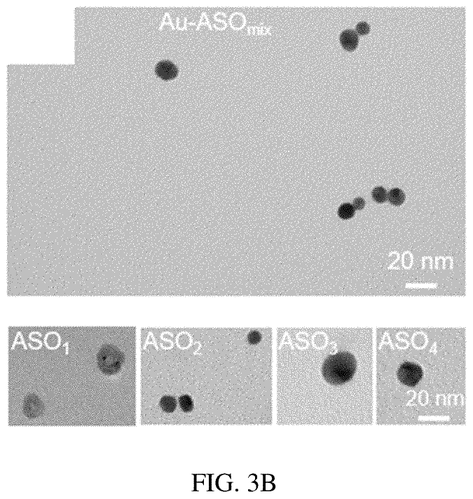

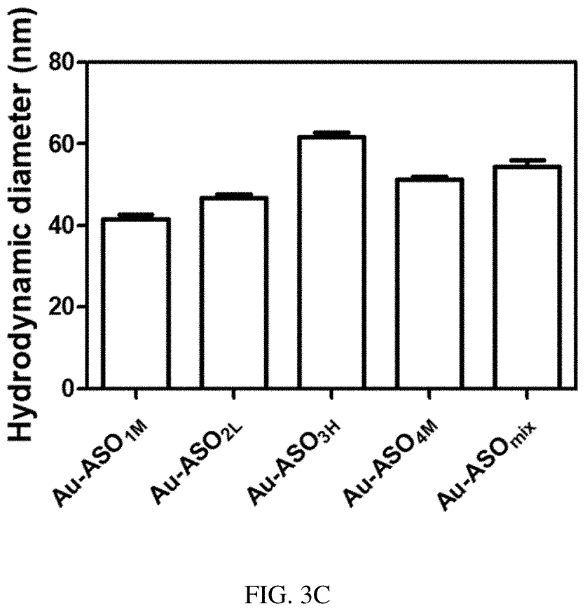

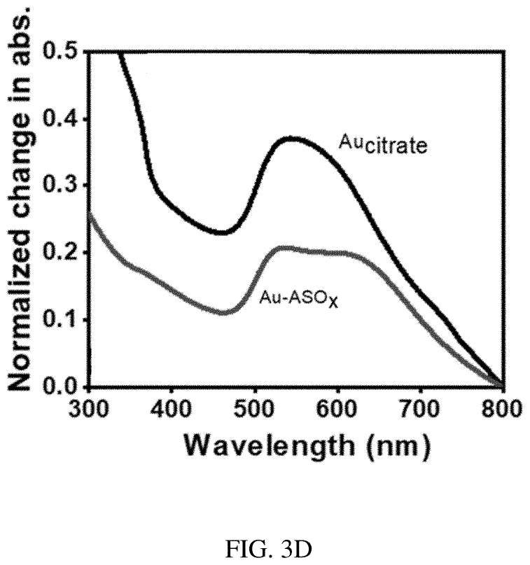

Accordingly, the differentially functionalized thiol modified ASOs from Example 1 were utilized to exchange the surface capping agent of the citrate stabilized gold nanoparticles. FIGS. 3A and 3B are transmission electron microscopy (TEM) images which show that the ASO capped AuNPs are individually dispersed with no visible aggregation. All the four ASO capped AuNPs exhibit a small anhydrous size of <30 nm, which are well dispersed without the formation of a large entity. FIG. 3C shows the average hydrodynamic sizes of the four individual ASO capped gold nanoparticles, which were found to be less than 60 nm. The formation of ASO conjugated thiol stabilized gold nanoparticles (AuNPs) was further confirmed from their surface plasmon bands. FIG. 3D shows two absorption peaks: one at ˜530 nm and the other at ˜620 nm was observed after the thiol modified ASO capping on the surface of the AuNPs.

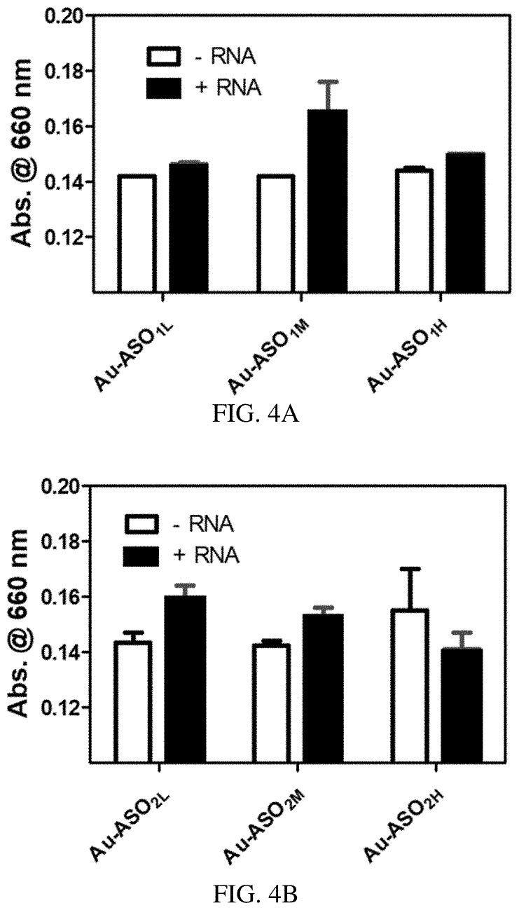

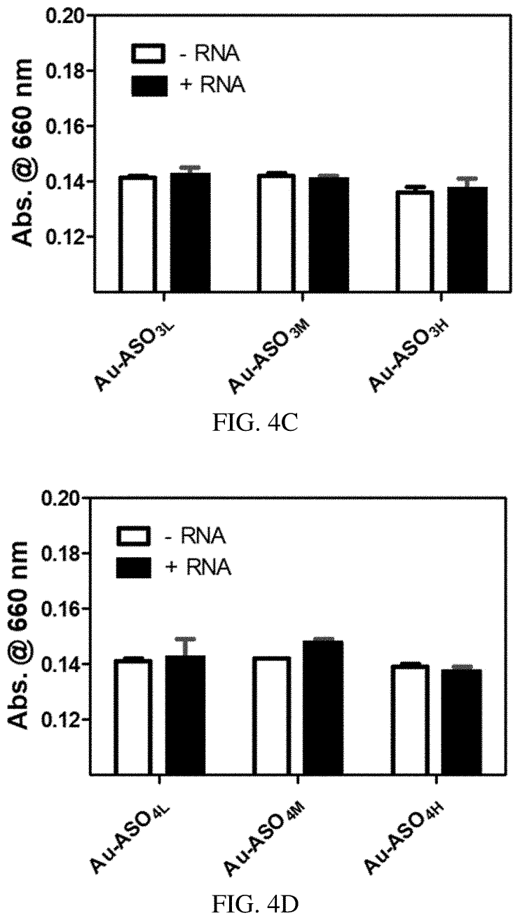

The relative sensitivity of the various ASO capped gold nanoparticles towards the target SARS-CoV-2 RNA was then monitored with the comparative increase in absorbance at 660 nm. It was observed that the surface capping of thiol modified ASOs together with the comparative ratio of the ASOs to the AuNPs (ASO/AuNPs) play a major role in determining the sensitivity of the gold nanoparticles towards SARS-CoV-2 RNA. To investigate the effect of the ASO/AuNPs ratio on the sensitivity of the sensing platform, three different ratios of ASO/AuNPs have been tested. The ratios are named as following: high (ASOH), Medium (ASOM), and low (ASOL) concentrations for the four ASOs, as described in Example 3. As shown in FIGS. 4A-4D, among these 12 different combinations of the ASO conjugated AuNPs, which vary in the ratio of ASO/AuNPs, ASOM was found the most sensitive ratio for the AuNPs capped with ASO1 (i.e. Au-ASO1M) as shown in FIG. 4A, while among ASO2, the low ratio was found to be the most sensitive in detecting the viral RNA (Au-ASO2L) as shown in FIG. 4B, the high ratio in ASO3 (Au-ASO3H) as shown in FIG. 4C, and finally the medium in ASO4 (Au-ASO4M) as shown in FIG. 4D.

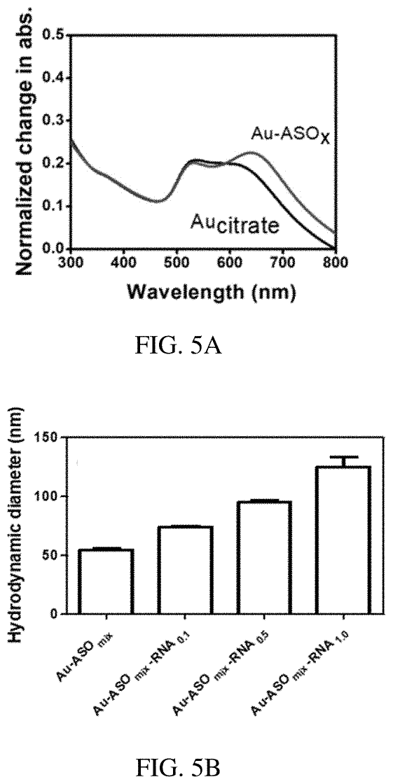

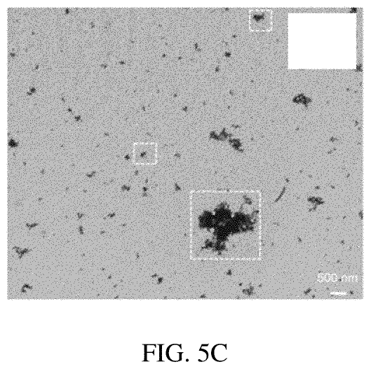

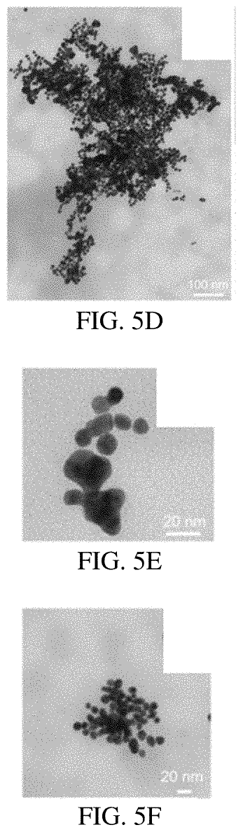

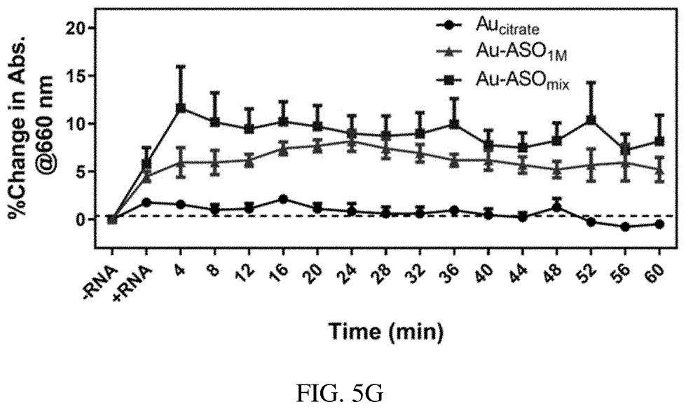

After optimizing the ASO to AuNPs ratio to obtain the maximum sensitivity, all the four ASO capped AuNPs (i.e. Au-ASO1M, Au-ASO2L, Au-ASO3H and Au-ASO4M) were mixed (Au-ASOmix) in an equivalent amount with each other to further improve the analytical sensitivity of the gold nanoparticles towards SARS-CoV-2 RNA. As shown in FIG. 3B transmission electron microscopy indicated the formation of distinctly dispersed AuNPs with the average hydrodynamic diameter of about 55.4±4.5 nm as observed from Zetaview, as shown in FIG. 3C. It was expected that the agglomeration propensity among the nanoparticles would increase when treated in a composite manner (Au-ASOmix) with the SARS-CoV-2 RNA. As shown in FIG. 5A large red-shift of about 40 nm in the aggregation band was also observed when the Au-ASOmix nanoparticles were tested against the SARS-CoV-2 RNA. The improvement in analytical sensitivity of Au-ASOmix nanoparticles towards the detection of SARS-CoV-2 viral RNA was further validated by monitoring the relative increase in absorbance at 660 nm in comparison with individual ASO capped AuNPs. The analytical performance of the sensor was also probed when two of the ASO capped AuNPs were mixed instead of all four (Au-ASOmix). The choice of two of the ASO capped AuNPs (either Au-ASO1M+2 L or Au-ASO3H+4M) was based on their proximity to target one of the regions of N gene sequence. In all cases, it was observed that Au-ASOmix was the optimum formulation to target SARS-CoV-2 RNA with higher sensitivity than the other sensors tested herein.

The Au-ASOmix nanoparticles were confirmed to be individually dispersed in the sample in absence of the viral load, which in presence of SARS-CoV-2 RNA tend to agglomerate forming large clusters. The aggregation of AuNPs was confirmed from the increase in absorbance of the aggregation band at 660 nm51-53 when the Au-ASOmix nanoparticles were exposed to a definite concentration of total RNA (1 ng/μL) extracted from the Vero cells infected with SARS-CoV-2 with an incubation time of 15 minutes at room temperature. As shown in FIG. 3C, it was evident that there was minimal change in hydrodynamic diameters when the individual ASO capped AuNPs were mixed to each other. But, as shown in FIG. 5B, the hydrodynamic diameter of Au-ASOmix nanoparticles increased largely with the addition of its target RNA containing SARS-CoV-2. This indicated the enhanced propensity of the nanoparticles to aggregate in presence of its target RNA containing SARS-CoV-2.

This response of Au-ASOmix nanoparticles to its target viral RNA was further corroborated by the TEM images. As shown in FIGS. 5C, 5D, 5E, and 5F, significant amount of clustering was found among the nanoparticles in presence of their target viral RNA. The formation of both large (˜120 nm) and small (˜80 nm) gold nanoparticle entities were observed in the sample. As shown in FIG. 5G, it was also monitored that the optimum sensitivity was achieved within 4 to 6 minutes of incubation at room temperature of the nanoparticles with the total RNA (1 ng/μL) extracted from the Vero cells infected with SARS-CoV-2.

### Example 5. Cell Culture, Isolation of RNA

Cercopithecus aethiops kidney epithelial cells (Vero E6) were procured from ATCC (CRL-1586™) and cultured at standard conditions in Eagle's Minimum Essential Medium with the supplement of 10% fetal bovine serum at 37° C. The cells were trypsinized with 0.25% (w/v) Trypsin—0.53 mM EDTA solution while maintaining the culture.

Severe acute respiratory syndrome-related coronavirus (SARS-CoV-2), isolate USA-WA1/2020 was isolated from an oropharyngeal swab of a patient with a respiratory illness. The patient had returned from travel to the affected region of China and developed clinical disease (COVID-19) in January 2020 in Washington, USA. The sample, NR-52287, as obtained from BEI Resources, NIAID, NIH, consists of a crude preparation of cell lysate and supernatant from Cercopithecus aethiops kidney epithelial cells (Vero E6; ATCC® CRL-1586™) infected with severe acute respiratory syndrome-related coronavirus 2 (SARS-CoV-2), isolate USA-WA1/2020 that was gamma-irradiated (5×106 RADs) on dry ice. The sample, NR-50549, as obtained from BEI Resources, NIAID, NIH, consists of a gamma irradiated cell lysate and supernatant from Vero cells infected with MERS-CoV, EMC/2012. This sample was isolated from a man with pneumonia in Saudi Arabia.

The Vero cells with or without the viral transfection were lysed directly in a culture dish by adding 1 mL of TRIzol reagent and aspirated carefully. The total RNA was then extracted and purified for the viral RNA from the cellular lysate with a commercially available kit. The concentration of purified RNA, isolated from the SARS-CoV-2 infected Vero cells, was found to be 35.9 ng/μL, while the concentration of the purified RNA, isolated from the non-infected Vero cells, was 92.6 ng/μL.

### Example 6. Sample Standardization, Signal Amplification Protocols, and RNAse H Treatment

The as-synthesized nanoparticles were taken out from the refrigerator, sonicated for 5 minutes in a bath sonicator (Branson 2800) at room temperature and vortexed for 2 minutes prior use. To determine the sensing capability of the individually ASO capped gold nanoparticles, the as-synthesized solution containing the AuNPs were treated with RNA samples having the concentration of 1 ng/μL. For the preparation of each 100 μL of Au-ASOmix, 25 μL of each individual Au-ASO1M, Au-ASO2L, Au-ASO3H and Au-ASO4M nanoparticles were mixed and vortexed thoroughly. The sensing and targeting capability of the Au-ASOmix was also validated at an RNA concentration of 1 ng/μL.

The signal amplification was investigated following a literature reported protocol. Briefly, 100 μL solution of Au-ASOmix was first treated with RNA containing SARS-CoV-2 at a concentration of 1 ng/μL. This solution was then incubated with cetyltrimethylammonium bromide (CTAB), L-ascorbic acid and chloroauric acid (HAuCl4) at a concentration of 0.1 M, 0.45 mM and 0.225 mM respectively and monitored over several time points.

Thermostable RNase H was purchased from New England Biolabs. For each 100 μL of reaction, 10 μL of RNase H reaction buffer (1×) was used along with 1 μL of thermostable RNase H and incubated for required time at a definite temperature. The 100 μL solution of Au-ASOmix nanoparticle was first treated with RNA samples having 1 ng/μL concentration and then incubated with RNase H reaction buffer (1×) and thermostable RNase H for required time points and temperature.

### Example 7. Measurement of Absorbance Spectra, Measurement of Hydrodynamic Diameter, Transmission Electron Microscopy, Hyperspectral Analysis, and Readout Methodology

The absorbance spectra were initially acquired on a VWR UV-Vis spectrophotometer, while the assays with 96 well plate was monitored on Biotek Synergy Neo2 Microplate Reader both for endpoint, kinetic and spectral analyses.

The hydrodynamic diameters of the individually ASO capped gold nanoparticles and the composite nanoparticles (Au-ASOmix) were monitored on a particle tracking analyzer (Zetaview Particle Metrix). The hydrodynamic diameters of Au-ASOmix before and after the addition of RNA at a concentration of 1 ng/μL were also observed in a similar fashion. The as-synthesized nanoparticles were diluted 50 times and 1 mL of such diluted samples were injected into the machine for the measurements. The chamber of the machine was properly cleaned prior each measurement.

The as-synthesized nanoparticles, Au-ASOmix before and after the addition of RNA at a concentration of 1 ng/μL were investigated under the transmission electron microscope (FEI tecnai T12). The tungsten filament was used as the electron optics and the voltage was kept constant at 80 kV. A 20 μL sample droplet was spotted onto a carbon-coated copper grid (400 mesh) and allowed to stay there for about 10 minutes before being removed.

### Example 8. Selectivity of Au-ASOₘᵢₓ Toward SARS-CoV-2

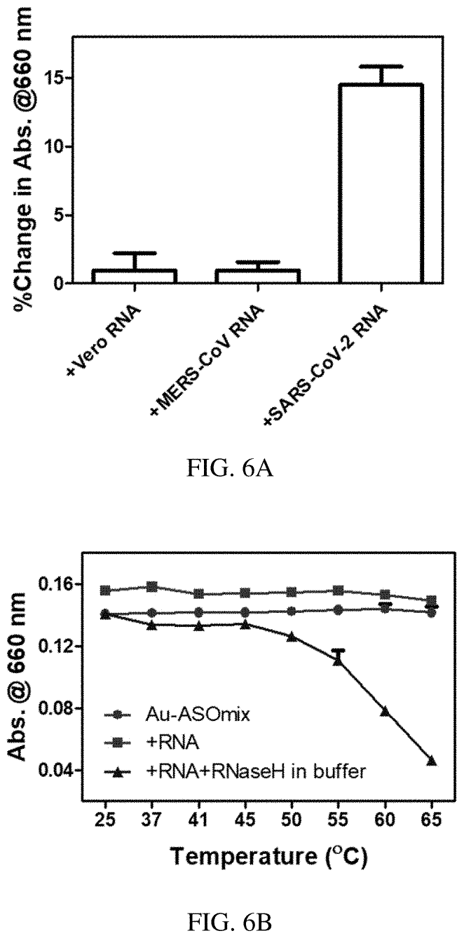

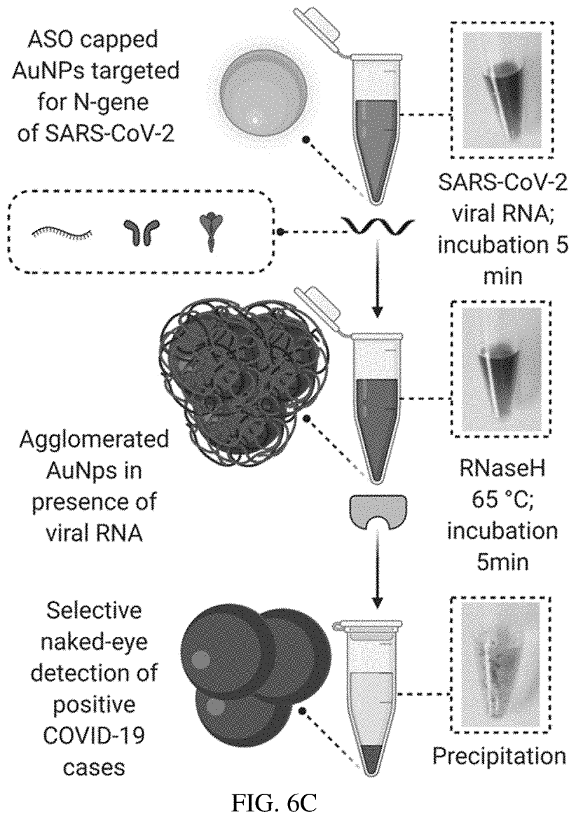

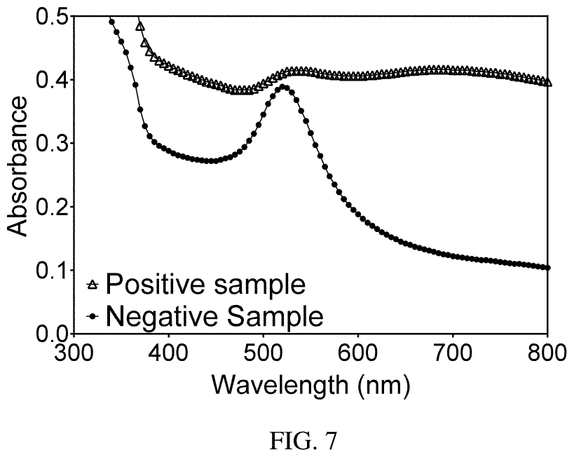

The selectivity of the SARS-CoV-2 sensor from Examples 1 through 7 was tested when the Au-ASOmix nanoparticle was treated against the total RNA isolated from cell lysate of Vero cells infected with MERS-CoV and the total RNA isolated from cell lysate of non-infected Vero cells. As shown in FIG. 6A, an insignificant change in absorbance at 660 nm wavelength was observed when the Au-ASOmix nanoparticles were treated with these RNAs (1 ng/4). Thus, both the RNAs extracted from non-infected Vero cells and Vero cells infected with MERS-CoV acted as negative controls for our experiments. Thus, the bio-engineered ASO capped gold nanoparticles, Au-ASOmix, could potentially be used for the selective detection of SARS-CoV-2. Selectivity of ASO1 and ASO2 were also demonstrated separately, as shown in FIG. 7, which shows a significant shift of the gold nanoparticle plasmonic peak at about 520 nm in the presence of SARS-CoV-2, confirming aggregation of the gold nanoparticles.

### Example 9. Colorimetric Change and Visual Detection of SARS-CoV-2

During the previous set of experiments under Example 8, an increase in absorbance at 660 nm wavelength with a red shift of ˜40 nm was observed with a difference in color from violet to dark blue, but a marked change in visual appearance was desired to potentially be used for the detection of SARS-CoV-2.

A bioassay was evaluated with the addition of thermostable RNase H to the mixture containing Au-ASOmix and total RNA having the SARS-CoV-2 gene. It has been envisaged that the thermostable RNase H will specifically recognize and cleave the phosphodiester bonds of the SARS-CoV-2 RNA (N gene) strand hybridized with the Au-ASOmix nanoconjugate while leaving the ASO strands intact. It has been presumed that this treatment of RNase H may greatly influence the agglomeration propensity among the gold nanoparticles those are already hybridized along the RNA strand which might also fulfill our aim of achieving an immediate change in visual appearance of the solution. To our expectation, no change in absorbance at 660 nm from the base absorbance of Au-ASOmix nanoparticle was observed when the hybridized Au-ASOmix nanoconjugate with SARS-CoV-2 RNA was treated with RNase H with an incubation of 5 minutes at room temperature. FIG. 6B shows that a further decrease in absorbance was observed when the mixture was incubated at higher temperatures indicating the increased activity of RNase H at elevated temperature levels. A marked change in visual appearance of the solution was achieved when the mixture was incubated at an elevated temperature of 65° C. for 5 minutes, which is schematically represented in FIG. 6C. This phenomenon may therefore be explained from the activity of RNase H to selectively cleave the RNA strand from the RNA conjugate with Au-ASOmix which leads to further agglomeration among the AuNPs those are attached to the RNA strand followed by precipitation of AuNPs from the solution. The test was also found to be selective to the presence of viral SARS-CoV-2 RNA load as the treatment of RNase H to the sample containing Au-ASOmix and RNA from Vero cells infected with MERS-CoV, caused no change in absorbance. Overall, this study reports the ‘naked-eye’ detection of COVID-19 causative virus, SARS-CoV-2, within a minimal timeframe of about 10 minutes from the total RNA derived from the virus infected cells.

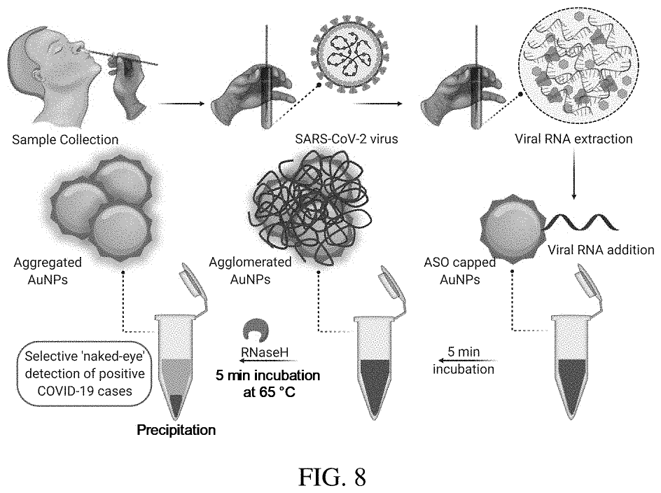

FIG. 8 shows a schematic representation for one embodiment the selective “naked-eye” detection of SARS-CoV-2 RNA, mediated by ASO capped gold nanoparticles, utilizing techniques as described herein, including as described in Examples 1, 2, 3, 6, and 9. In the embodiment shown in FIG. 8, a clinical sample is collected from a patient which contains SARS-CoV-2 virus. Viral RNA is extracted from the sample, and the sample is incubated with ASO capped gold nanoparticles designed and functionalized as described in Examples 1, 2, and 3, after which the sample is incubated with RNase H for 5 minutes at 65° C. as described in Example 9, resulting in the precipitation of aggregated gold nanoparticles, allowing for naked-eye detection of clinical samples which are positive for SARS-CoV-2 virus.

### Example 10. RNA Extraction-Free Sample Processing for Nucleic Acid Amplification-Based System

We subsequently also investigated the performance of the nanoparticle-based system with direct sampling, i.e., using an RNA extraction-free-approach. Guanidinium isothiocyanate lysis buffer at a sample:lysis buffer:water ratio of 2:1:2 was used for direct sample testing. We used this sample preparation condition due to its superior results compared to other tested sample preparation methods.

### Example 11. Design of Nucleic Acid Amplification Primers for RT-LAMP

We targeted the nucleocapsid phosphoprotein (N) gene sequence (1260 bp) of severe acute respiratory syndrome coronavirus 2 isolates (2019-nCoV/USA-WA1-A12/2020), the accession number is MT020880 in the NCBI repository. The nucleotide sequence of the N-gene ranges from 28274-29533 and the nucleotides selected for designing NAA primers were 28321-29520. These 1200 nucleotide bases were added to the PrimerExplorer v.5 (https://primerexplorer.jp/e/) software to design NAA primers suitable for the current NACT method22. In our previous publication, we identified four unique antisense oligonucleotides (ASOs) targeting particular segments of the N gene6,7. We observed that two of the developed ASOs were specific for 28695-28736 (ASO1 and ASO2). The binding energy of this pair (i.e., ASO1 and ASO2) was found to be highest among other predicted pairs. Thus, NAA primers were designed to target the region covered by ASO1 and ASO2. Primers are typically designed with melting temperatures between 54 and 67° C. Five NAA primer sets were generated which varied in their start and end positions within the targeted region. Among the generated 5 primer sets, we selected the set having dG=−2.36, which targets the 28525-28741 range of N gene sequence. dG is the reaction's disassociation constant, which measures the strength or spontaneity of dimerizing.

RT-LAMP is a one-pot isothermal NAA method that first converts RNA to DNA followed by amplification of cDNA in the same reaction pot. This NAA method therefore utilizes three pairs of primers and enzymes for nucleic acid amplification in a single tube. Briefly, the conversion of RNA to cDNA first takes place by the action of the reverse transcriptase enzyme. Four primers (the forward inner primer (FIP), backward inner primer (BIP), forward outer primer (F3), and outer backward primer (B3)) are then used to target four different regions of the gene, Bst DNA polymerase is used to amplify nucleic acid under isothermal conditions, while the other two primers, known as “loop primers” are used to accelerate the NAA reaction. Thus, six primers are used under isothermal conditions to amplify a single target gene in this NAA technique, in contrast to RT-PCR where primarily two primers are used at varying temperature conditions. Although this NAA produces many amplicons with various structural conformations instead of one major amplified genetic segment like RT-PCR, it has other advantages like the use of cost-effective equipment, high levels of sensitivity and amplification efficiency compared to RT-PCR71. Herein, F3, B3, FIP and BIP sequences were identified to amplify the region targeted by ASO1 and ASO2. Forward (LF) and backward (LB) loop primers were also designed from these primers. A primer set consisting of six primers has thus been designed for NAA, which are specific for SARS-CoV-2 N gene segment as shown in Table 2. Primers can be designed to make the protocol applicable to any viral or bacterial sequence by altering the initial target nucleotide sequence. Currently, as a proof-of-concept, we have established the method to target N gene sequence of COVID-19 causative virus, SARS-CoV-2. The successful amplification of the SARS-CoV-2 RNA using this approach has been confirmed using gel electrophoresis, as shown in FIG. 9, which shows the produced amplicons are of various structural conformations. The lanes of laddered banding pattern indicate positive amplification and spurious amplification products generated during the RT-LAMP reaction. The NAA was carried out with primer concentrations as follows: FIP (16 μM), BIP (16 μM), F3 (2 μM), B3 (2 μM), Loop F (4 μM), and Loop B (4 μM).

TABLE 2
NAA primer sequences to target the N gene of SARS-CoV-2

| Label | Type | Sequence (5′-3′) |
|---|---|---|
| F3 | Forward outer | TGGCTACTACCGAAGAGCT |
| | | (SEQ ID NO 10) |
| B3 | Reverse outer | TGCAGCATTGTTAGCAGGAT |
| | | (SEQ ID NO 11) |
| FIP | Forward inner (F1c*-F2) | TCTGGCCCAGTTCCTAGGTAGT-GACGAATTCGTGGTGGTGA |
| | | (SEQ ID NO 12) |
| BIP | Reverse inner (B1c*-B2) | AGACGGCATCATATGGGTTGCA-GCGGGTGCCAATGTGATC |
| | | (SEQ ID NO 13) |
| LF | Forward loop | TGGACTGAGATCTTTCATTTTACC |
| | | (SEQ ID NO 14) |
| LB | Reverse loop | ACTGAGGGAGCCTTGAATACACCA |
| | | (SEQ ID NO 15) |

*F1c is the complementary to F1 and B1c is the complementary sequence to B1.

### Example 12. Nucleic Acid Amplification Protocol

The mixture of nucleic acid amplification primers was prepared as set out in Table 3 (10×):

TABLE 3
Mixture of NAA primers mix (10X)

| Component | | Final concentration (μM) | Amount (μl) |
|---|---|---|---|
| FIP | (100 μM) | 16 | 32 |
| BIP | (100 μM) | 16 | 32 |
| F3 | (100 μM) | 2 | 4 |
| B3 | (100 μM) | 2 | 4 |
| Loop F | (100 μM) | 4 | 8 |
| Loop B | (100 μM) | 4 | 8 |

| | |
|---|---|
| RNAse free water | 112 |

Nucleic acid amplification reaction mixture was prepared in a 1.5 mL Eppendorf or PCR tube as set out in Table 4 (enough for four replicates):

TABLE 4
Nucleic Acid Amplification reaction mixture

| Component | Amount (μl) |
|---|---|
| WarmStart LAMP 2X Master Mix | 5 |
| LAMP Primer Mix (10X) | 1 |
| RNase free water | 2 |
| Total volume | 8 |

WarmStart technology was used to inhibit activity of the NAA reagents at room temperature. Briefly, the kit includes WarmStart LAMP 2× Master Mix, which contains a blend of Bst 2.0 WarmStart DNA Polymerase and WarmStart RTx Reverse Transcriptase in an optimized LAMP buffer solution.

8 μL aliquots of the NAA reaction mixture from Table 3 were prepared, which were added to 2 μL of collected sample (sample can be either extracted RNA or direct sample treated with lysis buffer or artificial saliva sample spiked with clinical samples' RNA) in PCR strip tubes or PCR plates. Samples were mixed well by pipetting up and down for few times. Water was used as a negative control.

### Example 13. Qualitative and Semiquantitative Detection of SARS-Cov-2

100 μL of an Au-ASOmix test solution including ASO1 and ASO2, prepared according to Example 6, was added to the tube containing the 10 μL of the amplified gene. The mixture was heated at 65° C. for 5 minutes. A color change can be observed with the naked eye, and/or by a plate reader. In the latter case, the mixture was transferred to a 96-well-plate and the color change was spectroscopically investigated by reading the absorbance spectrum at 660 nm.

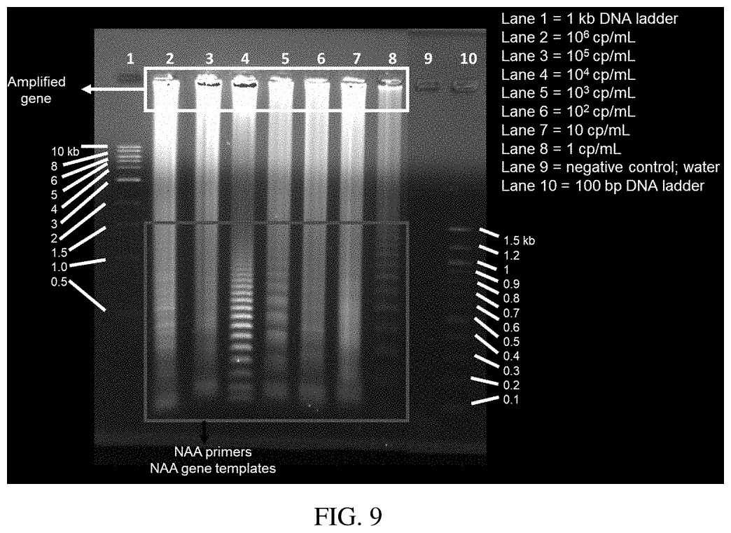

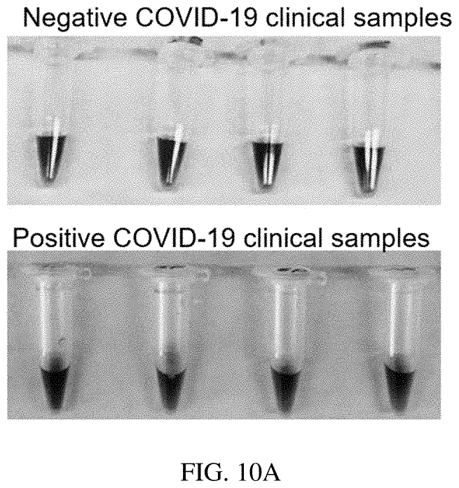

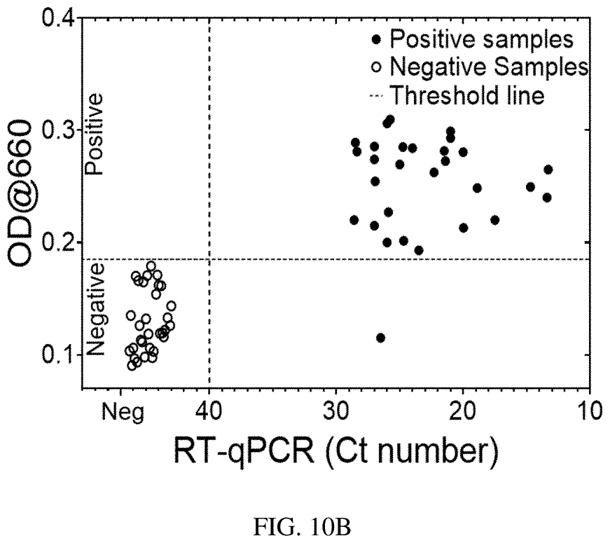

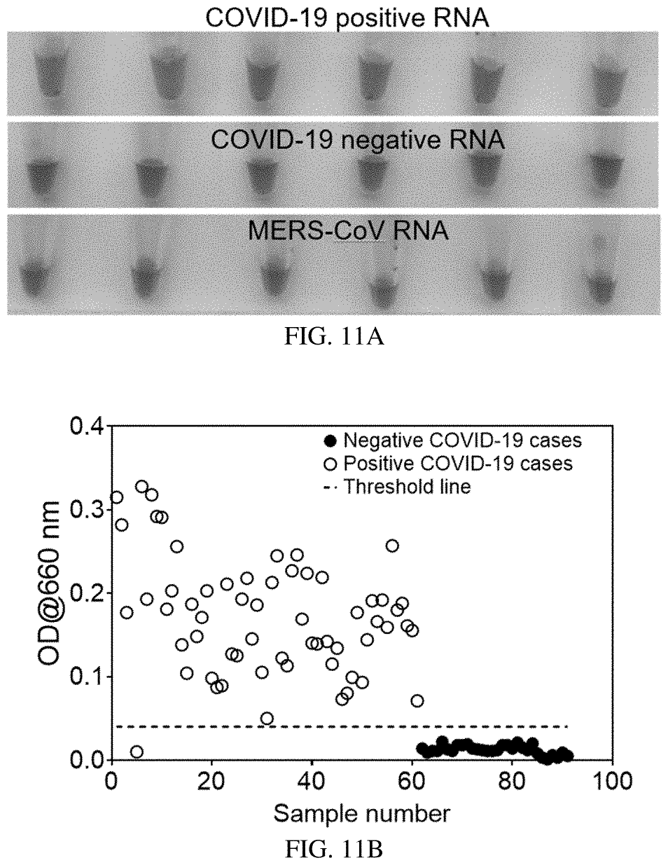

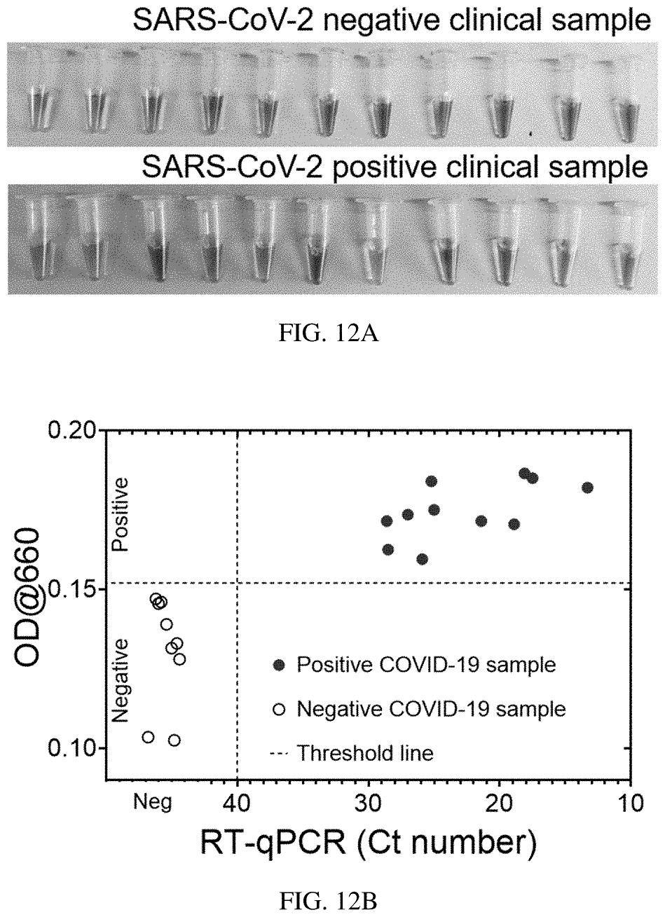

The visual readout of the test can be monitored by the naked eye, as shown in FIGS. 10A, 11A and 12A. Further, the assay can be easily performed in a multi-well plate format and the absorbance can be recorded using a conventional plate reader, as shown in FIGS. 10B, 11B, and 12B. The assay results can be also monitored using a handheld readout (e.g. portable plate reader). FIG. 12 depicts a “clear color change” that takes place in positive viral samples when samples are used directly, without RNA extraction. To avoid any ambiguity in interpreting the color of the sample, the protocol can also use a quantitative absorption measurement to avoid any subjectivity in the analysis.

The first line of COVID-19 management is to separate and quarantine the infected people from their surroundings at the earliest possibility. Therefore, it is sufficient to distinguish between the presence and absence of SARS-CoV-2 RNA and thus identify the infected samples. Thus, the qualitative detection of COVID-19 is far more important than the quantitative detection of the viral load in the infected samples. However, the aggregation of ASO-capped AuNPs leading to their change in surface plasmon bands and change in UV-Visible absorbance can be quantitative. However, the color change of the solution can only be used to provide a Yes/No answer. Like most naked-eye colorimetric approaches, the system described in Examples 10-13 also depends on the user's observation and color perception to draw inference on the diagnosis of the sample (i.e., positive or negative). Therefore, the introduction of a plasmonic optical readout will further eliminate the subjectivity of the test and avoid potential errors due to the variation in color interpretation from person to person. Additionally, the test could be made quantitative by establishing a standard curve that correlates the viral RNA quantity with an optical readout.

Although herein we have represented our data only in terms of increase in absorbance at 660 nm wavelength, as shown in FIGS. 10B, 11B, and 12B, there is a significant red shift in the absorbance spectra of the gold nanoparticles in presence of SARS-CoV-2 RNA. A shift of ˜40 nm was observed in the aggregation band of ASO conjugated AuNPs only in presence of the SARS-CoV-2 RNA as shown in Examples 7 and 8. Hence, there will be no problem in discriminating between the positive and negative samples by the naked eye. In combination with other detection methods demonstrated in Examples 7 and 8, these results point towards a multi-particle aggregation phenomenon caused due to the intermolecular hydrogen bonding among the nucleotides which also influences the agglomeration among multiple gold nanoparticles to form a clustered assembly.

### Example 14. Adaptability of the System to Test for Other Pathogens

In order to adapt the protocol described in Examples 10 through 13 to detect other biological targets, the following steps could be used.

Suitable antisense oligonucleotide (ASO) sequences need to be designed that specifically target RNA of the disease-causing virus/pathogen. Particular attention should be paid to designing the ASO sequences since their proximity will influence the agglomeration of the nanoparticles leading to a color change.

Next, the inner, outer and loop primer sequences need to be altered to amplify the target gene sequence to which the ASOs will hybridize. Overall, by changing the sequences of ASOs and primers according to the principles described in the previously described Examples, the capability of the protocol could be expanded to other pathogens.

Suitable standardization of lysis buffers must be performed while utilizing this technology for the detection of other targets. The membranes of different microorganisms comprise varied biological materials and hence might behave differently in different types of lysis buffer. Therefore, to obtain an optimized result, one needs to standardize the lysis conditions for each target as was done during the development of this protocol for SARS-CoV-2.

Basic knowledge in molecular biology is required to design ASOs and NAA primers. Basic knowledge of material science and chemistry are also recommended to perform the methods demonstrated in the Examples described herein, including the synthesis of AuNPs and conjugating them with ASOs. Once all the reagents and required materials are prepared, performing the test to detect a biological pathogen is quite straightforward. As the system, compositions and methods described herein offers the possibility of biological pathogen detection even without the extraction of RNA, it enables individuals with limited scientific expertise to conduct the test.

### Example 15. Detection of Hepatitis C Virus

Following the methods set out in Example 1 and the principles of Example 14, ASOs were identified specific to Hepatitis C Virus (HCV). Specifically, two ASOs were selected, targeted towards (i) stem-loop structure within the 5′ noncoding region (5′-NCR) known to be important for internal ribosome entry site (IRES) function (ASO5) and (ii) sequences spanning the AUG used for initiation of HCV polyprotein translation (ASO6). The sequences of these ASOs are represented below in Table 5.

TABLE 5
Sequences of the HCV targeting ASOs

| | Sequence (5′-3′) |
|---|---|
| ASO5 | HS-C6-GCCTTTCGCGACCCAACACT (SEQ ID NO 16) |
| ASO6 | HS-06-GTGCTCATGGTGCACGGTCT (SEQ ID NO 17) |

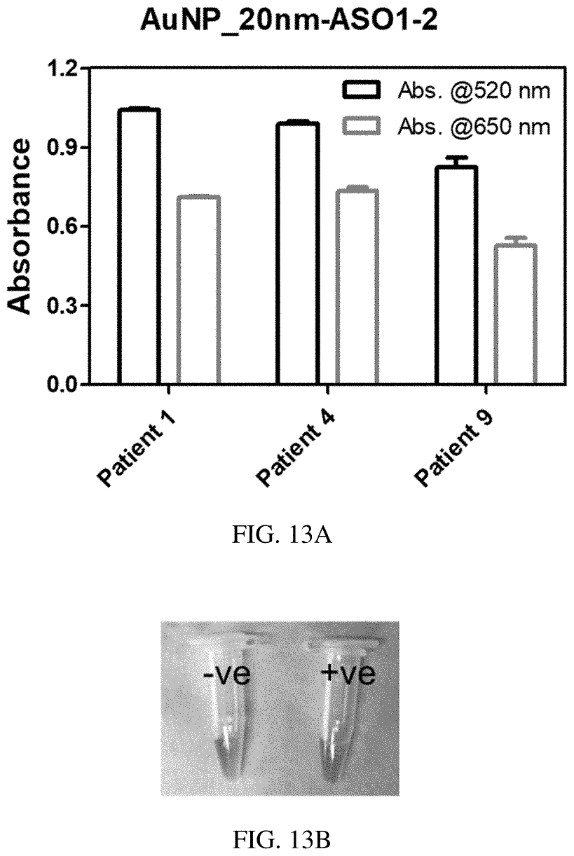

These thiolated ASOs were then used to cap citrate stabilized gold nanoparticles. The blood samples were drawn from patients under investigation for HCV infection. Plasma was isolated from the blood samples and treated with lysis buffer for RNA isolation. Without any further RNA extraction and purification steps, these lysis buffer treated plasma samples were added to the gold nanoparticle suspensions and change in absorbance was recorded. It was observed that there is a significant increase in absorbance only in presence of HCV positive plasma samples. A representative graph with three plasma samples has been shown in FIG. 13A. Change in visual color of the solution was also obtained in presence of HCV positive plasma samples, as shown in FIG. 13B. The current limit of detection of the technology is 55 IU/μL which can be lowered by 10⁶ times when integrated with nucleic acid amplification methodology. The plasmonic data were further compared with the data obtained from RT-PCR and tabulated below in Table 6.

TABLE 6
Comparison of plasmonic and RT-PCR data for detection of HCV

| | | Determined viral load | |
|---|---|---|---|
| Clinical | Presenceof | RT-PCR | Plasmonic |
| plasma | HCV | (IU/mL) | Technology (IU/mL) |
| Patient 1 | Yes | ~3,361,226 | ~3,294,002 |
| Patient 2 | Yes | ~1,697,755 | ~1,646,822 |
| Patient 3 | Yes | ~866,020 | ~840,039 |
| Patient 4 | Yes | ~3,361,226 | ~3,260,389 |
| Patient 5 | Yes | ~1,697,755 | ~1,663,799 |
| Patient 6 | Yes | ~34285 | ~32571 |

| | | |
|---|---|---|
| Patients 7-12 | No | — |

### Example 16. Specific Detection of Influenza H1N1

Following the methods set out in Example 1 and the principles of Example 14, ASOs were identified specific to Influenza A H1N1. Specifically, two ASOs were selected, targeted towards the HA gene of Influezna A H1N1 (ASO 7 and ASO 8). The sequences of these ASOs are represented below in Table 7.

TABLE 7
Sequences of the Influenza A H1N1 targeting ASOs

| | Target Sequence (5′-3′) | ASO Sequence (5′-3′) |
|---|---|---|
| ASO7 | CUAGUACUGUGUCUACAGUGUC | GACACTGTAGACACAGTACTAG |
| | (SEQ ID NO 18) | (SEQ ID NO 20) |
| ASO8 | ACAGGAAGCAAAGCACAGGG | CCCTGTGCTTTGCTTCCTGT |
| | (SEQ ID NO 19) | (SEQ ID NO 21) |

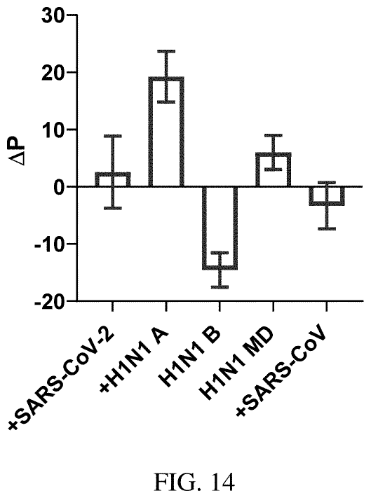

Another important parameter for any biosensor is the selectivity of the sensor toward its target. In this regard, the selectivity of the current Influenza A H1N1 sensor was tested when the Au-ASO mix comprising ASO7 and ASO8 was treated against the total RNA isolated from cell lysate of Vero cells infected with MERS-CoV and RNA of Influenza B H1N1, Influenza A H1N1 Maryland strain, SARS-CoV, and SARS-CoV-2. As shown in FIG. 14, the sensor only showed a high response in the presence of the RNA of its target the Influenza A H1N1 virus, and an insignificant change in signal was observed in the presence of the other samples.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A composition for use in the detection of a biological pathogen in a sample, the composition comprising:
a) a plurality of first anti-sense oligonucleotides, functionalized with a thiol moiety at their 5′ ends, the sequence of which is complementary to a first nucleic acid sequence in a target gene of the biological pathogen;
b) a plurality of second anti-sense oligonucleotides, functionalized with a thiol moiety at their 3′ ends, the sequence of which is complementary to a second nucleic acid sequence in the target gene of the biological pathogen near to the first nucleic acid sequence; and
c) a plurality of plasmonic nanoparticles capable of covalently binding to the thiol moieties;
wherein upon the first and second anti-sense oligonucleotides binding to the first and second nucleic acid sequences in the target gene respectively, the plasmonic nanoparticles covalently bind to the thiol moieties on the first and second anti-sense oligonucleotides respectively and are brought within proximity of one another and agglomerate.

2. The composition of claim 1, further comprising:
a) a plurality of third anti-sense oligonucleotide, functionalized with a thiol moiety at its 5′ end, the sequence of which is complementary to a third nucleic acid sequence in the target gene of the biological pathogen; and
b) a plurality fourth anti-sense oligonucleotide, functionalized with a thiol moiety at its 3′ end, the sequence of which is complementary to a fourth nucleic acid sequence in the target gene of the biological pathogen near to the third nucleic acid sequence;
wherein the third and fourth nucleic acid sequences are distant from the first and second nucleic acid sequences in the target gene, and wherein upon the third and fourth anti-sense oligonucleotides binding to the third and fourth nucleic acid sequences in the target gene respectively, the plasmonic nanoparticles covalently bind to the thiol moieties on the third and fourth anti-sense oligonucleotides and are brought within proximity of one another and agglomerate.

3. The composition of claim 2, wherein the biological pathogen is SARS-CoV-2, and wherein the sequences of the first, second, third and fourth anti-sense oligonucleotides are SEQ ID NO 6, SEQ ID NO 7, SEQ ID NO 8, and SEQ ID NO 9, respectively.

4. The composition of claim 1, wherein the plasmonic nanoparticles are gold nanoparticles.

5. The composition of claim 1, wherein the first and second anti-sense oligonucleotides have an unpaired probability for the first and second nucleic acid sequence respectively of at least 0.5.

6. The composition of claim 1, wherein the first and second anti-sense oligonucleotides have a binding of energy of less than −8 kcal/mol.

7. The composition of claim 1, wherein the first and second anti-sense oligonucleotides are present in differing ratios relative to the plasmonic nanoparticles.

8. A diagnostic apparatus for the detection of a biological pathogen in a clinical sample, comprising:
a) a container for a clinical sample in solution;
b) a plurality of first anti-sense oligonucleotides, functionalized with a thiol moiety at their 5′ ends, the sequence of which is complementary to a first nucleic acid sequence in a target gene of the biological pathogen;
c) a plurality of second anti-sense oligonucleotides, functionalized with a thiol moiety at their 3′ ends, the sequence of which is complementary to a second nucleic acid sequence in the target gene of the biological pathogen near to the first nucleic acid sequence; and
d) a plurality of plasmonic nanoparticles capable of covalently binding to the thiol moieties;
wherein if the clinical sample contains the biological pathogen, upon mixing the first and second anti-sense oligonucleotides and plasmonic nanoparticles with the clinical sample in solution, the first and second anti-sense oligonucleotides bind to the first and second nucleic acid sequences in the target gene respectively, and the plasmonic nanoparticles covalently bind to the thiol moieties on the first and second anti-sense oligonucleotides respectively and are brought within proximity of one another and agglomerate.

9. The apparatus of claim 8, further comprising:
a) a plurality of third anti-sense oligonucleotide, functionalized with a thiol moiety at their 5′ ends, the sequence of which is complementary to a third nucleic acid sequence in the target gene of the biological pathogen; and
b) a plurality fourth anti-sense oligonucleotide, functionalized with a thiol moiety at their 3′ ends, the sequence of which is complementary to a fourth nucleic acid sequence in the target gene of the biological pathogen near to the third nucleic acid sequence;
wherein the third and fourth nucleic acid sequences are distant from the first and second nucleic acid sequences in the target gene, and wherein if the clinical sample contains the biological pathogen, upon mixing the third and fourth anti-sense oligonucleotides and plasmonic nanoparticles with the clinical sample in solution, the third and fourth anti-sense oligonucleotides bind to the third and fourth nucleic acid sequences in the target gene respectively, and the plasmonic nanoparticles covalently bind to the thiol moieties on the third and fourth anti-sense oligonucleotides and are brought within proximity of one another and agglomerate.

10. The apparatus of claim 9, wherein the biological pathogen is SARS-CoV-2, and wherein the sequences of the first, second, third and fourth anti-sense oligonucleotides are SEQ ID NO 6, SEQ ID NO 7, SEQ ID NO 8, and SEQ ID NO 9, respectively.

11. The apparatus of claim 8, wherein the plasmonic nanoparticles are gold nanoparticles.

12. The apparatus of claim 8, wherein the agglomeration is detectable through a color change in the solution, further comprising a colorimeter to detect the color change.

13. The apparatus of claim 8, further comprising a nuclease enzyme, wherein when the nuclease enzyme is mixed with the anti-sense oligonucleotides and plasmonic nanoparticles with a clinical sample containing the biological pathogen in solution, the nuclease enzyme cleaves the hybrid anti-sense oligonucleotide and nucleic acid sequence from the remainder of the target gene, resulting in the agglomeration and precipitation of covalently bound plasmonic nanoparticles from solution.

14. The apparatus of claim 8, wherein the first and second anti-sense oligonucleotides are present in differing ratios relative to the plasmonic nanoparticles.

15. A method for detecting a biological pathogen in a clinical sample, the method comprising:
a) collecting a clinical sample in solution;
b) mixing a plurality of first anti-sense oligonucleotides, functionalized with a thiol moiety at their 5′ ends, the sequence of which is complementary to a first nucleic acid sequence in a target gene of the biological pathogen, with the clinical sample in solution;
c) mixing a plurality of second anti-sense oligonucleotides, functionalized with a thiol moiety at their 3′ ends, the sequence of which is complementary to a second nucleic acid sequence in the target gene of the biological pathogen near to the first nucleic acid sequence, with the clinical sample in solution; and
d) mixing a plurality of plasmonic nanoparticles capable of covalently binding to the thiol moieties with the clinical sample in solution;
wherein if the clinical sample contains the biological pathogen, upon mixing the first and second anti-sense oligonucleotides and plasmonic nanoparticles with the clinical sample in solution, the first and second anti-sense oligonucleotides bind to the first and second nucleic acid sequences in the target gene respectively, and the plasmonic nanoparticles covalently bind to the thiol moieties on the first and second anti-sense oligonucleotides respectively and are brought within proximity of one another and agglomerate.

16. The method of claim 15, further comprising:
a) mixing a plurality of third anti-sense oligonucleotide, functionalized with a thiol moiety at their 5′ ends, the sequence of which is complementary to a third nucleic acid sequence in the target gene of the biological pathogen, with the clinical sample in solution; and
b) mixing a plurality fourth anti-sense oligonucleotide, functionalized with a thiol moiety at their 3′ ends, the sequence of which is complementary to a fourth nucleic acid sequence in the target gene of the biological pathogen near to the third nucleic acid sequence, with the clinical sample in solution;
wherein the third and fourth nucleic acid sequences are distant from the first and second nucleic acid sequences in the target gene, and wherein if the clinical sample contains the biological pathogen, upon mixing the third and fourth anti-sense oligonucleotides and plasmonic nanoparticles with the clinical sample in solution, the third and fourth anti-sense oligonucleotides bind to the third and fourth nucleic acid sequences in the target gene respectively, and the plasmonic nanoparticles covalently bind to the thiol moieties on the third and fourth anti-sense oligonucleotides and are brought within proximity of one another and agglomerate.

17. The method of claim 16, wherein the biological pathogen is SARS-CoV-2, and wherein the sequences of the first, second, third and fourth anti-sense oligonucleotides are SEQ ID NO 6, SEQ ID NO 7, SEQ ID NO 8, and SEQ ID NO 9, respectively.

18. The method of claim 15, wherein the plasmonic nanoparticles are gold nanoparticles.

19. The method of claim 15, further comprising performing nucleic acid amplification of the clinical sample in solution prior to the mixing of the plurality of first anti-sense oligonucleotides therewith.

20. The method of claim 19, wherein the nucleic acid amplification is performed by loop-mediated isothermal amplification.


SEQUENCE LISTING (21 sequences)

SEQ ID NO: 1 - DNA, 1380 bp, SARS-CoV-2 virus
  misc_feature: Target N gene sequence of SARS-CoV-2 isolate 2019-nCoV/USA-WA1-A12/2020
cgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgc

SEQ ID NO: 2 - RNA, 20 nt, SARS-CoV-2 virus
acaccaaaagaucacauugg

SEQ ID NO: 3 - RNA, 20 nt, SARS-CoV-2 virus
cccgcaauccugcuaacaau

SEQ ID NO: 4 - RNA, 20 nt, SARS-CoV-2 virus
cagaacaaacccaaggaaau

SEQ ID NO: 5 - RNA, 20 nt, SARS-CoV-2 virus
acugauuacaaacauuggcc

SEQ ID NO: 6 - DNA, 20 bp, Artificial Sequence
  Antisense oligonucleotide 1 (ASO1)
ccaatgtgatcttttggtgt

SEQ ID NO: 7 - DNA, 20 bp, Artificial Sequence
  Antisense oligonucleotide 2 (ASO2)
attgttagcaggattgcggg

SEQ ID NO: 8 - DNA, 20 bp, Artificial Sequence
  Antisense oligonucleotide 3 (ASO3)
atttccttgggtttgttctg

SEQ ID NO: 9 - DNA, 20 bp, Artificial Sequence
  Antisense oligonucleotide 4 (ASO4)
ggccaatgtttgtaatcagt

SEQ ID NO: 10 - DNA, 19 bp, Artificial Sequence
  Forward outer primer for nucleic acid amplification (NAA) testing
tggctactaccgaagagct

SEQ ID NO: 11 - DNA, 20 bp, Artificial Sequence
  Reverse outer primer for nucleic acid amplification (NAA) testing
tgcagcattgttagcaggat

SEQ ID NO: 12 - DNA, 41 bp, Artificial Sequence
  Forward inner primer for nucleic acid amplification (NAA) testing
tctggcccagttcctaggtagtgacgaattcgtggtggtga

SEQ ID NO: 13 - DNA, 40 bp, Artificial Sequence
  Reverse inner primer for nucleic acid amplification (NAA) testing
agacggcatcatatgggttgcagcgggtgccaatgtgatc

SEQ ID NO: 14 - DNA, 24 bp, Artificial Sequence
  Forward loop primer for nucleic acid amplification (NAA) testing
tggactgagatctttcattttacc

SEQ ID NO: 15 - DNA, 24 bp, Artificial Sequence
  Reverse loop primer for nucleic acid amplification (NAA) testing
actgagggagccttgaatacacca

SEQ ID NO: 16 - DNA, 20 bp, Artificial Sequence
  Antisense oligonucleotide 5 (ASO5)
gcctttcgcgacccaacact

SEQ ID NO: 17 - DNA, 20 bp, Artificial Sequence
  Antisense oligonucleotide  (ASO6)
gtgctcatggtgcacggtct

SEQ ID NO: 18 - RNA, 22 nt, Influenza A H1N1 virus
cuaguacugugucuacaguguc

SEQ ID NO: 19 - RNA, 20 nt, Influenza A H1N1 virus
acaggaagcaaagcacaggg

SEQ ID NO: 20 - DNA, 22 bp, Artificial Sequence
  Antisense oligonucleotide 7 (ASO7)
gacactgtagacacagtactag

SEQ ID NO: 21 - DNA, 20 bp, Artificial Sequence
  Antisense oligonucleotide 8 (ASO8)
ccctgtgctttgcttcctgt